(19) United States
(12) Patent Application Publication
(10) Pub. No.: US 2004/0152715 A1
(43) Pub. Date: Aug. 5, 2004

(54) Cycloalkylene amide compounds as NR2B receptor antagonists

(76) Inventors: Makoto Kawai, Aichi-ken (JP); Hiroshi Nakamura, Aichi-ken (JP); Hiroshia Shimokawa, Aichi-ken (JP)

Correspondence Address: WARNER-LAMBERT COMPANY, 2800 PLYMOUTH RD, ANN ARBOR, MI, 48105, US

(21) Appl. No.: 10/737,309

(22) Filed: Dec. 16, 2003

Related U.S. Application Data
(60) Provisional application No. 60/434,361, filed on Dec. 17, 2002.

(51) Int. Cl. A61K 31/505; C07D 213/72; A61K 31/44

(52) U.S. Cl. 514/275; 514/352; 544/330; 546/306

(57) ABSTRACT

This invention provides a compound of the formula (I):

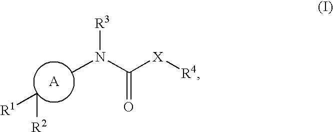

wherein R¹ represents

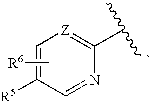

wherein R⁵ represents a hydroxy group or the like; R⁶ and R⁷ independently represents a hydrogen atom or the like: V represents an alkylene or the like: W represents a carbon atom or the like: Z represents a carbon or the like:

R² represents a hydrogen atom or the like: R³ represents a hydrogen or the like: A represents a cycloalkylene or the like: X represents a covalent bond or the like: R⁴ represents an aryl or the like. These compounds are useful for the treatment of disease conditions caused by overactivation of NMDA NR2B receptor such of pain, or the like in mammalian. This invention also provides a pharmaceutical composition comprising the above compound.

This application is a United States utility application, which claims the benefit of priority to U.S. provisional application Serial No. 60/434,361 filed Dec. 17, 2002.

### TECHNICAL FIELD

This invention relates to novel cycloalkylene amide compounds. These compounds are useful as antagonists of NMDA (N-methyl-D-aspartate) NR2B receptor, and are thus useful for the treatment of pain, stroke, traumatic brain injury, Parkinson's disease, Alzheimer's disease, depression, anxiety, migraine, or the like in mammalian, especially humans. The present invention also relates to a pharmaceutical composition comprising the above compounds.

### BACKGROUND ART

Glutamate plays a dual role in the central nervous system (CNS) as essential amino acid and the principal excitatory neurotransmitters. There are two major class of receptors, ionotoropic and metabotropic. Ionotropic receptors are classified into three major subclass, N-methyl-asparatate (NMDA), 2-amino-3(methyl-3-hydroxyisoxazol-4-yl)propionic acid (AMPA), kainate. There is considerable preclinical evidence that hyperalgesia and allodynia following peripheral tissue or nerve injury is not only due to an increase in the sensitivity of primary afferent nociceptors at the site of injury but also depends on NMDA receptor-mediated central changes in synaptic excitability. In humans, NMDA receptor antagonists have also been found to decrease both pain perception and sensitization. Also, overactivation of NMDA receptor is a key event for triggering neuronal cell death under pathological conditions of acute and chronic forms of neurodegeneration. However, while NMDA receptor inhibition has therapeutic utility in the treatment of pain and neurodegenerative diseases, there are significant liabilities to many available NMDA receptor antagonists that can cause potentially serious side effects. NMDA subunits are differentially distributed in the CNS. Especially, NR2B is believed to be restricted to the forebrain and laminas I and II of the dosal horn. The more discrete distribution of NR2B subunit in the CNS may support a reduced side-effect profile of agents that act selectively at this site.

For example, NMDA NR2B selective antagonists may have clinical utility for the treatment of neuropathic and other pain conditions in human with a reduced side-effect profile than existing NMDA antagonists (S. Boyce, et al., Neuropharmacology, 38, pp.611-623 (1999)).

Although heterocycloalkylene compounds synthesized are described in WO97/38665, it relates to inhibitors of farnesyl-protein taransferase. Further, WO0071516 and WO03/048158 disclose heterocyclic amide compounds, however they relate to inhibitors of factor Xa.

WO01/81295, EP982026, DE4437999, WO01/92239 and WO02/22592 disclose a variety of cycloalkylene amide compounds. In particular, Compound A, B C and D represented by the following formula are disclosed in WO 01/81295, EP 982026, DE 4437999 and WO02/22592 respectively.

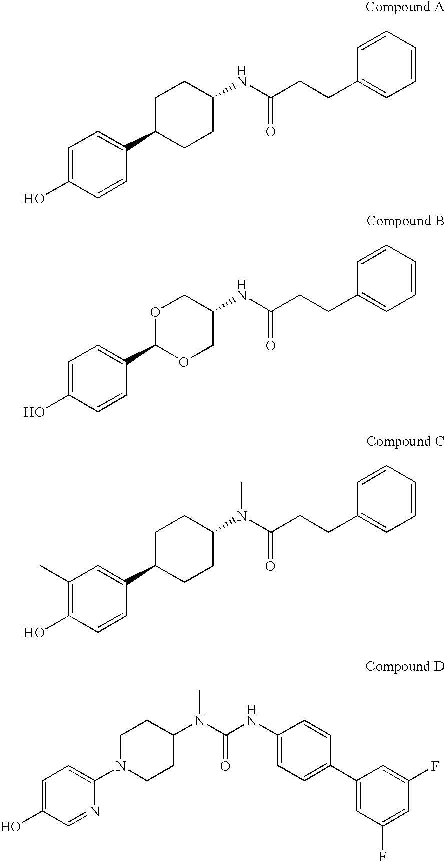

Compound C and D are not described as a NR2B antagonist in DE4437999 and WO02122592 respectively. Although both Compound A and B are NR2B receptor antagonists, the binding affinity of them are insufficient. Further, NR2B receptor antagonist activity of compound B is insufficient. Yet further, Compound A shows QT prolongation due to their potent inhibitory activity at HERG (human ether-a-go-go related gene) potassium channel. In the meantime, although Compound E described in WO 01/92239 shows a potent binding affinity, it shows QT prolongation due to the same reason mentioned above.

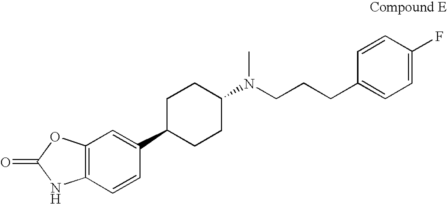

Therefore, it would be desirable if there were provided a novel NMDA NR2B selective antagonist with potent binding activity by systemic administration, and both with potent NR2Breceptor binding activity and with reduced inhibitory activity at HERG potassium channel.

### BRIEF DISCLOSURE OF THE INVENTION

It has now been found that cylcloalkylene amide compounds are NMDA NR2B selective antagonists with analgesic activity by systemic administration, and both with potent NR2Breceptor binding activity and with reduced inhibitory activity at HERG potassium channel. The cycloalkylene amide group at the ortho position of a nitrogen atom of the pyridine (or pyrimidine) ring and proton donor (e.g. a phenolic hydroxy group) at the para position of said cycloalkylene amide group resulted in a potent NMDA NR2B receptor antagonistic activity with analgesic activity by systemic administration, and both with potent NR2Breceptor binding activity and with reduced inhibitory activity at HERG potassium channel.

The compounds of the present invention may show less toxicity, good absorption, distribution, good solubility, low protein binding affinity, less drug-drug interaction, and good metabolic stability.

The present invention provides a compound of the following formula (I):

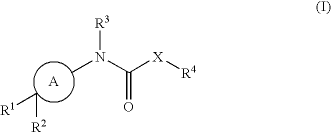

wherein R¹ represents

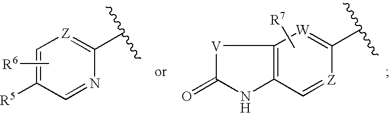

R⁵ represents a hydroxy group or an alkylsulfonylamino group having from 1 to 6 carbon atoms;

R⁶ and R⁷ independently represents a hydrogen atom, a halogen atom, an alkyl group having from 1 to 6 carbon atoms, an alkenyl group having from 2 to 6 carbon atoms, an alkoxy group having from 1 to 6 carbon atoms or, when Z represents a carbon atom and R⁶ is ortho to Z, R⁶ and Z taken together may form a fused phenyl group or a saturated or partially unsaturated cyclic ring having from 4 to 7 carbon atoms;

V represents an alkylene group having from 1 to 2 carbon atoms, imino, imino substituted with an alkyl group having from 1 to 6 carbon atoms, an oxygen atom or a sulfur atom;

W represents a carbon atom or a nitrogen atom;

Z represents a carbon atom or a nitrogen atom;

with the proviso that W and Z do not simultaneously represent a carbon atom;

R² represents a hydrogen atom or a hydroxy group or R² forms a covalent bond with ring A:

R³ represents a hydrogen atom or an alkyl group having from 1 to 6 carbon atoms:

A represents a cycloalkylene group having from 3 to 10 carbon atoms or a heterocyclic group having from 4 to 10 atoms;

X represents a covalent bond, an alkylene group having from 1 to 3 carbon atoms, an alkenylene group having from 2 to 3 carbon atoms, a heteroalkylene group having from 2 to 3 atoms, wherein one of said atoms is replaced by a sulfur atom, an oxygen atom, imino, imino substituted with an alkyl group having from 1 to 6 carbon atoms or a sulfonyl group, a cycloalkylene group having from 3 to 10 carbon atoms or a heterocyclic group having from 4 to 10 atoms;

R⁴ represents an aryl group having from 6 to 10 carbon atoms, a heteroaryl group having from 5 to 10 atoms;

said alkylene groups, alkenylene groups, heteroalkylene groups, cycloalkylene groups and heterocyclic groups are unsubstituted or are substituted by at least one substituent selected from the group consisting of substituents α;

said aryl groups having from 6 to 10 carbon atoms and said heteroaryl groups having from 5 to 10 atoms are unsubstituted or are substituted by at least one substituent selected from the group consisting of substituents β;

said substituents α are selected from the group consisting of alkyl groups having from 1 to 6 carbon atoms, cyano groups, alkanoylamino groups having from 1 to 7 carbon atoms, oxo groups or aryl groups having from 6 to 10 carbon atoms defined above;

said substituents β are selected from the atom consisting of halogen atoms, alkyl groups having from 1 to 6 carbon atoms, alkoxy groups having from 1 to 6 carbon atoms, haloalkyl groups having from 1 to 6 carbon atoms, alkylthio groups having from 1 to 6 carbon atoms, alkanoyl groups having from 1 to 7 carbon atoms, hydroxy groups, cyano groups, aryl groups having from 6 to 10 carbon atoms defined above or heteroaryl groups having from 5 to 10 atoms defined above;

with the proviso that said aryl groups having from 6 to 10 carbon atoms and said heteroaryl groups having from 5 to 10 atoms in said substituents α and β are not substituted by an aryl group having from 6 to 10 carbon atoms or heteroaryl groups having from 5 to 10 atoms; and

or a pharmaceutically acceptable ester of such compound;

or a pharmaceutically acceptable salt thereof.

The cycloalkylene amide compounds of this invention have an antagonistic action towards NMDA NR2B receptor subtype selectively and are thus useful in therapeutics, particularly for the treatment of stroke or brain injury, chronic neurodegenerative disease such as Parkinson's disease, Alzheimer's disease, Huntington's disease or amyotrophic lateral sclerosis (ALS), epilepsy, convulsive disorder, pain, anxiety, human immunodeficiency virus (HIV) related neuronal injury, migraine, depression, schizophrenia, tumor, post-anesthesia cognitive decline (PACD), glaucoma, tinnitus, tradive dyskinesia, allergic encephalomyelitis, opioid tolerance, drug abuse, alcohol abuse, Irritable bowel syndrome (IBS), or the like in mammalian, especially humans.

The compounds of the present invention are useful for the general treatment of pain, particularly neuropathic pain. Physiological pain is an important protective mechanism designed to warn of danger from potentially injurious stimuli from the external environment. The system operates through a specific set of primary sensory neurones and is exclusively activated by noxious stimuli via peripheral transducing mechanisms (Millan 1999 Prog. Neurobio. 57: 1-164 for an integrative Review). These sensory fibres are known as nociceptors and are characterised by small diameter axons with slow conduction velocities. Nociceptors encode the intensity, duration and quality of noxious stimulus and by virtue of their topographically organised projection to the spinal cord, the location of the stimulus. The nociceptors are found on nociceptive nerve fibres of which there are two main types, A-delta fibres (myelinated) and C fibres (non-myelinated). The activity generated by nociceptor input is transferred after complex processing in the dorsal horn, either directly or via brain stem relay nuclei to the ventrobasal thalamus and then on to the cortex, where the sensation of pain is generated.

Intense acute pain and chronic pain may involve the same pathways driven by pathophysiological processes and as such cease to provide a protective mechanism and instead contribute to debilitating symptoms associated with a wide range of disease states. Pain is a feature of many trauma and disease states. When a substantial injury, via disease or trauma, to body tissue occurs the characteristics of nociceptor activation are altered. There is sensitisation in the periphery, locally around the injury and centrally where the nociceptors terminate. This leads to hypersensitivity at the site of damage and in nearby normal tissue. In acute pain these mechanisms can be useful and allow for the repair processes to take place and the hypersensitivity returns to normal once the injury has healed. However, in many chronic pain states, the hypersensitivity far outlasts the healing process and is normally due to nervous system injury. This injury often leads to maladaptation of the afferent fibres (Woolf & Salter 2000 Science 288: 1765-1768). Clinical pain is present when discomfort and abnormal sensitivity feature among the patient's symptoms. Patients tend to be quite heterogeneous and may present with various pain symptoms. There are a number of typical pain subtypes: 1) spontaneous pain which may be dull, burning, or stabbing; 2) pain responses to noxious stimuli are exaggerated (hyperalgesia); 3) pain is produced by normally innocuous stimuli (allodynia) (Meyer et al., 1994 Textbook of Pain 13-44). Although patients with back pain, arthritis pain, CNS trauma, or neuropathic pain may have similar symptoms, the underlying mechanisms are different and, therefore, may require different treatment strategies. Therefore pain can be divided into a number of different areas because of differing pathophysiology, these include nociceptive, inflammatory, neuropathic pain etc. It should be noted that some types of pain have multiple aetiologies and thus can be classified in more than one area, e.g. Back pain, Cancer pain have both nociceptive and neuropathic components.

Nociceptive pain is induced by tissue injury or by intense stimuli with the potential to cause injury. Pain afferents are activated by transduction of stimuli by nociceptors at the site of injury and sensitise the spinal cord at the level of their termination. This is then relayed up the spinal tracts to the brain where pain is perceived (Meyer et al., 1994 Textbook of Pain 13-44). The activation of nociceptors activates two types of afferent nerve fibres. Myelinated A-delta fibres transmitted rapidly and are responsible for the sharp and stabbing pain sensations, whilst unmyelinated C fibres transmit at a slower rate and convey the dull or aching pain. Moderate to severe acute nociceptive pain is a prominent feature of, but is not limited to pain from strains/sprains, post-operative pain (pain following any type of surgical procedure), posttraumatic pain, burns, myocardial infarction, acute pancreatitis, and renal colic. Also cancer related acute pain syndromes commonly due to therapeutic interactions such as chemotherapy toxicity, immunotherapy, hormonal therapy and radiotherapy. Moderate to severe acute nociceptive pain is a prominent feature of, but is not limited to, cancer pain which may be tumour related pain, (e.g. bone pain, headache and facial pain, viscera pain) or associated with cancer therapy (e.g. postchemotherapy syndromes, chronic postsurgical pain syndromes, post radiation syndromes), back pain which may be due to herniated or ruptured intervertabral discs or abnormalities of the lumber facet joints, sacroiliac joints, paraspinal muscles or the posterior longitudinal ligament.

Neuropathic pain is defined as pain initiated or caused by a primary lesion or dysfunction in the nervous system (IASP definition). Nerve damage can be caused by trauma and disease and thus the term ‘neuropathic pain’ encompasses many disorders with diverse aetiologies. These include but are not limited to, Diabetic neuropathy, Post herpetic neuralgia, Back pain, Cancer neuropathy, HIV neuropathy, Phantom limb pain, Carpal Tunnel Syndrome, chronic alcoholism, hypothyroidism, trigeminal neuralgia, uremia, or vitamin deficiencies. Neuropathic pain is pathological as it has no protective role. It is often present well after the original cause has dissipated, commonly lasting for years, significantly decreasing a patients quality of life (Woolf and Mannion 1999 Lancet 353: 1959-1964). The symptoms of neuropathic pain are difficult to treat, as they are often heterogeneous even between patients with the same disease (Woolf & Decosterd 1999 Pain Supp. 6: S141-S147; Woolf and Mannion 1999 Lancet 353: 1959-1964). They include spontaneous pain, which can be continuous, or paroxysmal and abnormal evoked pain, such as hyperalgesia (increased sensitivity to a noxious stimulus) and allodynia (sensitivity to a normally innocuous stimulus).

The inflammatory process is a complex series of biochemical and cellular events activated in response to tissue injury or the presence of foreign substances, which result in swelling and pain (Levine and Taiwo 1994: Textbook of Pain 45-56). Arthritic pain makes up the majority of the inflammatory pain population. Rheumatoid disease is one of the commonest chronic inflammatory conditions in developed countries and rheumatoid arthritis is a common cause of disability. The exact aetiology of RA is unknown, but current hypotheses suggest that both genetic and microbiological factors may be important (Grennan & Jayson 1994 Textbook of Pain 397-407). It has been estimated that almost 16 million Americans have symptomatic osteoarthritis (OA) or degenerative joint disease, most of whom are over 60 years of age, and this is expected to increase to 40 million as the age of the population increases, making this a public health problem of enormous magnitude (Houge & Mersfelder 2002 Ann Pharmacother. 36: 679-686; McCarthy et al., 1994 Textbook of Pain 387-395). Most patients with OA seek medical attention because of pain. Arthritis has a significant impact on psychosocial and physical function and is known to be the leading cause of disability in later life. Other types of inflammatory pain include but are not limited to inflammatory bowel diseases (IBD),

Other types of pain include but are not limited to;

Musculo-skeletal disorders including but not limited to myalgia, fibromyalgia, spondylitis, sero-negative (non-rheumatoid) arthropathies, non-articular rheumatism, dystrophinopathy, Glycogenolysis, polymyositis, pyomyositis.

Central pain or ‘thalamic pain’ as defined by pain caused by lesion or dysfunction of the nervous system including but not limited to central post-stroke pain, multiple sclerosis, spinal cord injury, Parkinson's disease and epilepsy.

Heart and vascular pain including but not limited to angina, myocardical infarction, mitral stenosis, pericarditis, Raynaud's phenomenon, scleredoma, scleredoma, skeletal muscle ischemia.

Visceral pain, and gastrointestinal disorders. The viscera encompasses the organs of the abdominal cavity. These organs include the sex organs, spleen and part of the digestive system. Pain associated with the viscera can be divided into digestive visceral pain and non-digestive visceral pain. Commonly encountered gastrointestinal (GI) disorders include the functional bowel disorders (FBD) and the inflammatory bowel diseases (IBD). These GI disorders include a wide range of disease states that are currently only moderately controlled, including—for FBD, gastro-esophageal reflux, dyspepsia, the irritable bowel syndrome (IBS) and functional abdominal pain syndrome (FAPS), and—for IBD, Crohn's disease, ileitis, and ulcerative colitis, which all regularly produce visceral pain. Other types of visceral pain include the pain associated with dysmenorrhea, pelvic pain, cystitis and pancreatitis.

Head pain including but not limited to migraine, migraine with aura, migraine without aura cluster headache, tension-type headache.

Orofacial pain including but not limited to dental pain, temporomandibular myofascial pain.

The present invention provides a pharmaceutical composition for the treatment of disease conditions caused by overactivation of NMDA NR2B receptor, in a mammalian subject, which comprises administering to said subject a therapeutically effective amount of a compound of formula (I).

Further, the present invention also provides a composition which comprises a therapeutically effective amount of the cycloalkylene amide compound of formula (I) or its pharmaceutically acceptable salt together with a pharmaceutically acceptable carrier. Among them, the composition is preferably for the treatment of disease defined above.

Also, the present invention provides for the use of a compound of formula (I),

or a pharmaceutically acceptable ester of such compound, or a pharmaceutically acceptable salt thereof, as a medicament.

Also, the present invention provides a method for the treatment of disease conditions defined above, which comprises administering to said subject a therapeutically effective amount of a compound of formula (1).

Further, the present invention provides a method for the treatment of disease conditions defined above in a mammal, preferably human, which comprises administering to said subject a therapeutically effective amount of a compound of formula (I).

Yet further, the present invention provides the use of a therapeutically effective amount of a compound of formula (I) in the manufacture of a medicament for the treatment of the disease conditions defined above.

### DETAILED DESCRIPTION OF THE INVENTION

As used herein, the term “halogen” means fluoro, chloro, bromo and iodo, preferably fluoro or chloro.

As used herein, the term “alkyl” means straight or branched chain saturated radicals, including, but not limited to methyl, ethyl, n-propyl, isopropyl, n-butyl, iso-butyl, secondary-butyl, tertiary-butyl.

As used herein, the term “alkenyl” means a hydrocarbon radical having at least one double bond including, but not limited to, ethenyl, propenyl, 1-butenyl, 2-butenyl and the like.

As used herein, the term “alkoxy” means alkyl-O—, including, but not limited to methoxy, ethoxy, n-propoxy, isopropoxy, n-butoxy, iso-butoxy, secondary-butoxy, tertiary-butoxy.

As used herein, the term “ilnino” means —NH—.

As used herein, the term “alkanoyl” means a group having carbonyl such as R′—C(O)— wherein R′ is H, C₁₋₆ alkyl, phenyl or C₃₋₆ cycloalkyl, including, but not limited to formyl, acetyl, ethyl-C(O)—, n-propyl-C(O)—, isopropyl-C(O)—, n-butyl-C(O)—, iso-butyl-C(O)—, secondary-butyl-C(O)—, tertiary-butyl-C(O)—, cyclopropyl-C(O)—, cyclobutyl-C(O)—, cyclopentyl-C(O)—, cyclohexyl-C(O)—, and the like.

As used herein, the term “aryl” means a monocyclic or bicyclic aromatic carbocyclic ring of 6 to 10 carbon atoms; or bicyclic partially saturated carbocyclic ring of 6 to 10 carbon atoms including, but not limited to, phenyl, naphthyl, indanyl, indenyl, tetralinyl, preferably phenyl and naphthyl.

The term “alkylene”, as used herein, means a saturated hydrocarbon (straight chain or branched) wherein a hydrogen atom is removed from each of the terminal carbons such as methylene, ethylene, methylethylene, propylene, butylene, pentylene, hexylene and the like.

The term “alkenylene”, as used herein, means a hydrocarbon radical having at least one double bond (straight chain or branched) wherein a hydrogen atom is removed from each of the terminal carbons such as ethenylene, propenylene, and the like.

The term “heteroalkylene”, as used herein, means a saturated hydrocarbon radical having from 2 to 3 atoms, wherein one of said atoms is replaced by a sulfur atom, an oxygen atom, imino, imino substituted with an alkyl group having from 1 to 6 carbon atoms or a sulfonyl group; and wherein a hydrogen atom is removed from each of the terminal carbons such as C1-2 alkylene-O—, C1-2 alkylene-N—, C1-2 alkylene-S(O)ₙ— wherein n represents 0 to 2; methylene-O-methylene, methylene-N-methylene, methylene-S(O)ₙ-methylene wherein n represents 0 to 2; and the like.

The term “cycloalkylene”, as used herein, means a saturated or a partially saturated mono-or bi-carbocyclic radical ring of 3 to 10 carbon atoms; and wherein a hydrogen atom is removed from each of the terminal carbons, including, but not limited to, cyclopropylene, cyclobutylene, cyclopentylene, cyclohexylene, cyclohexenylene, cycloheptylene, cyclooctylene, cyclononylen, cyclodecylen, bicyclo[3.3.0]octylene, bicyclo[3.2.1]octylene, bicyclo[3.3.1]nonylene, and the like.

The term “heterocyclic group”, as used herein, means a 4 to 10-membered saturated, partially saturated ring, which consists of at least one carbon atom and from 1 to 4 heteroatoms independently selected from the atoms consisting of sulfur atoms, oxygen atoms and nitrogen atoms, and including any bicyclic group; and wherein a hydrogen atom is removed from each of the terminal carbons. Examples of such heterocycles include, but are not limited to, piperidine, 4-piperidone, pyrrolidine, 2-pyrrolidone, trahydrofurane, tetrahydroquinoline, tetrahydroisoquinoline, decahydroquinoline or octahydroisoquinoline, pyrrolidine, piperidine or piperazine.

The term “haloalkyl”, as used herein, means an alkyl radical which is substituted by halogen atoms as defined above including, but not limited to, fluoromethyl, difluoromethyl, trifluoromethyl, 2-fluoroethyl, 2,2-difluoroethyl, 2,2,2-trifluoroethyl, 2,2,2-trichloroethyl, 3-fluoropropyl, 4-fluorobutyl, chloromethyl, trichloromethyl, iodomethyl and bromomethyl groups and the like.

The term “heteroaryl” means a 5- to 10-membered aromatic or partially saturated hetero mono- or bi-cyclic ring which consists of from 1 to 4 heteroatoms independently selected from the group consisting of sulfur atoms, oxygen atoms and nitrogen atoms including, but not limited to, pyrazolyl, furyl, thienyl, oxazolyl, tetrazolyl, thiazolyl, imidazolyl, thiadiazolyl, pyridyl, pyrimidinyl, pyrrolyl, thiophenyl, pyrazinyl, pyridazinyl, isooxazolyl, isothiazolyl, triazolyl, furazanyl, indolinyl, benzothienyl, benzofuranyl, benzoimidazolinyl, quinolinyl, tetrahydroquinolinyl, and the like.

Where the compounds of formula (I) contain hydroxy groups, they may form esters. Examples of such esters include esters with a hydroxy group and esters with a carboxy group. The ester residue may be an ordinary protecting group or a protecting group which can be cleaved in vivo by a biological method such as hydrolysis.

The term “ordinary protecting group” means a protecting group, which can be cleaved by a chemical method such as hydrogenolysis, hydrolysis, electrolysis or photolysis.

The term “esters” means a protecting group which can be cleaved in vivo by a biological method such as hydrolysis and forms a free acid or salt thereof. Whether a compound is such a derivative or not can be determined by administering it by intravenous injection to an experimental animal, such as a rat or mouse, and then studying the body fluids of the animal to determine whether or not the compound or a pharmaceutically acceptable salt thereof can be detected.

Preferred examples of groups for an ester of a hydroxy group include: lower aliphatic acyl groups, for example: alkanoyl groups, such as the formyl, acetyl, propionyl, butyryl, isobutyryl, pentanoyl, pivaloyl, valeryl, isovaleryl, octanoyl, nonanoyl, decanoyl, 3-methylnonanoyl, 8-methylnonanoyl, 3-ethyloctanoyl, 3,7-dimethyloctanoyl, undecanoyl, dodecanoyl, tridecanoyl, tetradecanoyl, pentadecanoyl, hexadecanoyl, 1-methylpentadecanoyl, 14-methylpentadecanoyl, 13,13-dimethyltetradecanoyl, heptadecanoyl, 15-methylhexadecanoyl, octadecanoyl, 1-methylheptadecanoyl, nonadecanoyl, icosanoyl and henicosanoyl groups; halogenated alkylcarbonyl groups, such as the chloroacetyl, dichloroacetyl, trichloroacetyl, and trifluoroacetyl groups; alkoxyalkylcarbonyl groups, such as the methoxyacetyl group; and unsaturated alkylcarbonyl groups, such as the acryloyl, propioloyl, methacryloyl, crotonoyl, isocrotonoyl and (E)-2-methyl-2-butenoyl groups; more preferably, the lower aliphatic acyl groups having from 1 to 6 carbon atoms; aromatic acyl groups, for example: arylcarbonyl groups, such as the benzoyl, α-naphthoyl and β-naphthoyl groups; halogenated arylcarbonyl groups, such as the 2-bromobenzoyl and 4-chlorobenzoyol groups; lower alkylated arylcarbonyl groups, such as the 2, 4,6-trimethylbenzoyl and 4-toluoyl groups; lower alkoxylated arylcarbonyl groups, such as the 4-anisoyl group; nitrated arylcarbonyl groups, such as the 4-nitrobenzoyl and 2-nitrobenzoyl groups; lower alkoxycarbonylated arylcarbonyl groups, such as the 2-(methoxycarbonyl)benzoyl group; and arylated arylcarbonyl groups, such as the 4-phenylbenzoyl group; alkoxycarbonyl groups, for example: lower alkoxycarbonyl groups, such as the methoxycarbonyl, ethoxycarbonyl, propoxycarbonyl, butoxycarbonyl, sec-butoxycarbonyl, t-butoxycarbonyl and isobutoxycarbonyl groups; and halogen- or tri(lower alkyl)silyl-substituted lower alkoxycarbonyl groups, such as the 2,2,2-trichloroethoxycarbonyl and 2-trimethylsilylethoxycarbonyl groups; tetrahydropyranyl or tetrahydrothiopyranyl groups, such as: tetrahydropyran-2-yl, 3-bromotetrahydropyran-2-yl, 4-methoxytetrahydropyran-4-yl, tetrahydrothiopyran-2-yl, and 4-methoxytetrahydrothiopyran-4-yl groups; tetrahydrofuranyl or tetrahydrothiofuranyl groups, such as: tetrahydrofuran-2-yl and tetrahydrothiofuran-2-yl groups; silyl groups, for example: tri(lower alkyl)silyl groups, such as the trimethylsilyl, triethylsilyl, isopropyldimethylsilyl, t-butyldimethylsilyl, methyldiisopropylsilyl, methyldi-t-butylsilyl and triisopropylsilyl groups; and tri(lower alkyl)silyl groups substituted by 1 or 2 aryl groups, such as the diphenylmethylsilyl, diphenylbutylsilyl, diphenylisopropylsilyl and phenyldiisopropylsilyl groups; alkoxymethyl groups, for example: lower alkoxymethyl groups, such as the methoxymethyl, 1,1-dimethyl-1-methoxymethyl, ethoxymethyl, propoxymethyl, isopropoxymethyl, butoxymethyl and t-butoxymethyl groups; lower alkoxylated lower alkoxymethyl groups, such as the 2-methoxyethoxymethyl group; and halo(lower alkoxy)methyl groups, such as the 2,2,2-trichloroethoxymethyl and bis(2-chloroethoxy)methyl groups; substituted ethyl groups, for example: lower alkoxylated ethyl groups, such as the 1-ethoxyethyl and 1-(isopropoxy)ethyl groups; and halogenated ethyl groups, such as the 2,2,2-trichloroethyl group; aralkyl groups, for example: lower alkyl groups substituted by from 1 to 3 aryl groups, such as the benzyl, α-naphthylmethyl, β-naphthylmethyl, diphenylmethyl, triphenylmethyl, α-naphthyldiphenylmethyl and 9-anthrylmethyl groups; and lower alkyl groups substituted by from 1 to 3 substituted aryl groups, where one or more of the aryl groups is substituted by one or more lower alkyl, lower alkoxy, nitro, halogen or cyano substituents, such as the 4-methylbenzyl, 2,4,6-trimethylbenzyl, 3,4,5-trimethylbenzyl, 4-methoxybenzyl, 4-methoxyphenyldiphenylmethyl, 2-nitrobenzyl, 4-nitrobenzyl, 4-chlorobenzyl, 4-bromobenzyl and 4-cyanobenzyl groups; alkenyloxycarbonyl groups: such as the vinyloxycarbonyl and aryloxycarbonyl groups; and aralkyloxycarbonyl groups in which the aryl ring may be substituted by 1 or 2 lower alkoxy or nitro groups: such as the benzyloxycarbonyl, 4-methoxybenzyloxycarbonyl, 3,4-dimethoxybenzyloxycarbonyl, 2-nitrobenzyloxycarbonyl and 4-nitrobenzyloxycarbonyl groups.

The term “treating”, as used herein, refers to reversing, alleviating, inhibiting the progress of, or preventing the disorder or condition to which such term applies, or one or more symptoms of such disorder or condition. The term “treatment” as used herein refers to the act of treating, as “treating” is defined immediately above.

A preferred compound of formula (I) of this invention is that wherein R¹ represents formula (x)

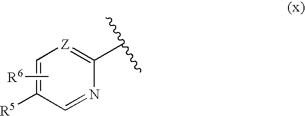

where Z represents preferably a carbon atom.

Where R¹ represents formula (x), R⁵ represents preferably a hydroxy group and R⁶ represents a hydrogen atom, a halogen atom or an alkyl group having from 1 to 6 carbon atoms, more preferably a hydrogen atom or a halogen atom, most preferably a hydrogen atom.

A most preferred compound of formula (I) of this invention is that wherein R¹ represents 5-hydroxy-pyridin-2-yl.

A preferred compound of formula (I) of this invention is that wherein R² represents a hydrogen atom or a hydroxy group.

A preferred compound of formula (I) of this invention is that wherein R³ represents a hydrogen atom or methyl, preferably hydrogen.

A preferred compound of formula (I) of this invention is that wherein A represents a substituted or unsubstituted cycloalkylene group having from 3 to 8 carbon atoms, more preferably 4 to 6 carbon atoms or A is an heterocyclic group having from 4 to 8 atoms which consists of at least one carbon atom and from 1 to 2 heteroatoms selected from the atoms consisting of sulfur atoms, oxygen atoms and nitrogen atoms, more preferably from 1 to 2 nitrogen atoms. Most preferably A represents a substituted or unsubstituted cyclohexyl group, a cyclohexenyl group or a piperidinyl group, preferably unsubstituted cyclohexyl.

A preferred compound of formula (I) wherein A is substituted is that wherein the substituent is at least one group selected from alkyl groups having from 1 to 6 carbon atoms, preferably 1 to 3 carbon atoms, or oxo groups. Most preferably, the substituent is at least one alkyl group having from 1 to 3 carbon atoms.

A preferred compound of formula (I) wherein X represents a covalent bond, a sulfonyl group or a substituted or unsubstituted alkylene group having from 1 to 3 carbon atoms, an alkenylene group having from 2 to 3 carbon atoms, a heteroalkylene group having from 2 to 3 atoms, wherein one of said atoms is replaced by a sulfur atom, an oxygen atom, imino, imino substituted with an alkyl group having from 1 to 6 carbon atoms. More preferably, X represents an alkylene group having from 1 to 3 carbon atoms, a heteroalkylene group having from 2 to 3 atoms, wherein one of said atoms is replaced by a sulfur atom or an oxygen atom. Yet more preferably X represents a substituted or unsubstituted alkylene group having from 1 to 3 carbon atoms, or a heteroalkylene group having from 2 to 3 atoms, wherein one of said atoms is replaced by a sulfur atom, most preferably an ethylene group or —CH₂S—.

A preferred compound of formula (I) wherein X is substituted is that wherein the substituent is at least one group selected from alkyl groups having from 1 to 6 carbon atoms or oxo groups, more preferably alkyl groups having from 1 to 6 carbon atoms.

A preferred group of formula (I) of this invention is that wherein R⁴ represents an unsubstituted or substituted aryl group having from 6 to 8 carbon atoms, preferably a phenyl group, or an unsubstituted or substituted a heteroaryl group having from 5 to 8 atoms. More preferably, R⁴ represents an unsubstituted or substituted phenyl group.

A preferred compound of formula (I) wherein R⁴ is substituted is that wherein the substituent is at least one group selected from halogen atoms, alkyl groups having from 1 to 6 carbon atoms, alkoxy groups having from 1 to 6 carbon atoms, haloalkyl groups having from 1 to 6 carbon atoms, alkylthio groups having from 1 to 6 carbon atoms, alkanoyl groups having from 1 to 6 carbon atoms, hydroxy groups or cyano groups. A further preferred compound of formula (I) wherein R⁴ is substituted is that wherein the substituent is at least one group selected from halogen atoms, alkyl groups having from 1 to 6 carbon atoms, alkoxy groups having from 1 to 6 carbon atoms, haloalkyl groups having from 1 to 6 carbon atoms or hydroxy groups. A most preferred compound of formula (I) wherein R⁴ is substituted is that wherein the substituent is one or more groups selected from halogen atoms, preferably chloro or fluoro, or alkyl groups having from 1 to 6 carbon atoms.

A most preferred compound of formula (I) is that wherein R⁴ is phenyl, optionally substituted by one or more halogen atoms, e.g. chloro or fluoro, or alkyl groups having from 1 to 6 carbon atoms, e.g. methyl.

A preferred group of formula (I) of this invention is that wherein the groups R¹ and —N(R³)— are in a trans relationship.

Particularly preferred compounds of the invention include those in which each variable in Formula (I) is selected from the preferred groups for each variable. Even more preferable compounds of the invention include those where each variable in Formula (I) is selected from the more preferred groups for each variable.

As a further aspect of the present invention, there is provided a compound of formula (Ia)

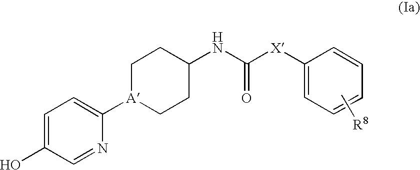

wherein

A′ represents CH, C(OH), or N;

X′ represents ethylene, oxymethylene, methyleneoxy, or methylenethio; and

R⁸ represents one or two groups independently selected from hydrogen atoms, alkyl groups having from 1 to 6 carbon atoms and halogen atoms

or a pharmaceutically acceptable ester of such compound;

or a pharmaceutically acceptable salt thereof.

A preferred individual compound of this invention is selected from

N-[cis-4-Hydroxy-4-(5-hydroxypyridin-2-yl)cyclohexyl]-3-phenylpropanamide hydrochloride;

3-(4-Chlorophenyl)-N-[cis-4-hydroxy-4-(5-hydroxypyridin-2-yl)cyclohexyl] propanamide;

N-[cis-4-Hydroxy-4-(5-hydroxypyridin-2-yl)cyclohexyl]-N-methyl-3-phenylpropanamide;

N-[trans-4-(5-Hydroxypyridin-2-yl)cyclohexyl]-3-phenylpropanamide hydrochloride;

N-[trans-4-(5-Hydroxypyridin-2-yl)cyclohexyl]-N-methyl-3-phenylpropanamide hydrochloride;

3-(2,4-dichlorophenyl)-N-[cis-4-hydroxy-4-(5-hydroxypyridin-2-yl)cyclohexyl]propanamide;s

N-[cis-4-hydroxy-4-(5-hydroxypyridin-2-yl)cyclohexyl]-3-(4-methylphenyl)propanamide;

3-(2-fluorophenyl)-N-[cis-4-hydroxy-4-(5-hydroxypyridin-2-yl)cyclohexyl]propanamide;

3-(2-fluorophenyl)-N-[trans-4-(5-hydroxypyridin-2-yl)cyclohexyl]propanamide;

3-(4-fluorophenyl)-N-[trans-4-(5-hydroxypyridin-2-yl)cyclohexyl]propanamide;

N-[trans-4-(5-hydroxypyridin-2-yl)cyclohexyl]-2-(phenylthio)acetamide;

3-(4-ethylphenyl)-N-[trans-4-(5-hydroxypyridin-2-yl)cyclohexyl]propanamide;

3-(2-chlorophenyl)-N-[trans-4-(5-hydroxypyridin-2-yl)cyclohexyl]propanamide

3-(4-chlorophenyl)-N-[trans-4-(5-hydroxypyridin-2-yl)cyclohexyl]propanamide;

3-(4-methylphenyl)-N-[trans-4-(5-hydroxypyridin-2-yl)cyclohexyl]propanamide;

3-(2-fluorophenyl)-N-[cis-4-hydroxy-4-(5-hydroxypyridin-2-yl)cyclohexyl]-N-methylpropanamide;

N-[4-(5-Hydroxypyridin-2-yl)cyclohex-3-en-1-yl]-3-phenylpropanamide;

2-fluorobenzyl [cis-4-hydroxy-4-(5-hydroxypyridin-2-yl)cyclohexyl]methylcarbamate;

benzyl [cis-4-hydroxy-4-(5-hydroxypyridin-2-yl)cyclohexyl]methylcarbamate;

3-(2-fluorophenyl)-N-[1-(5-hydroxypyridin-2-yl)piperidin-4-yl]propanamide; and

N-[1-(5-hydroxypyridin-2-yl)piperidin-4-yl]-3-(4-methylphenyl)propanamide;

or a pharmaceutically acceptable salts thereof.

A most preferred individual compound of this invention is selected from

N-[cis-4-Hydroxy-4-(5-hydroxypyridin-2-yl)cyclohexyl]-3-phenylpropanamide hydrochloride;

3-(4-Chlorophenyl)-N-[cis-4-hydroxy-4-(5-hydroxypyridin-2-yl)cyclohexyl] propanamide;

N-[trans-4-(5-Hydroxypyridin-2-yl)cyclohexyl]-3-phenylpropanamide hydrochloride;

3-(2-fluorophenyl)-N-[cis-4-hydroxy-4-(5-hydroxypyridin-2-yl)cyclohexyl]propanamide;

3-(2-fluorophenyl)-N-[trans-4-(5-hydroxypyridin-2-yl)cyclohexyl]propanamide;

3-(4-fluorophenyl)-N-[trans-4-(5-hydroxypyridin-2-yl)cyclohexyl]propanamide; and

N-[trans-4-(5-hydroxypyridin-2-yl)cyclohexyl]-2-(phenylthio)acetamide;

or a pharmaceutically acceptable salt thereof.

### General Synthesis

The compounds of the present invention may be prepared by a variety of processes well known for the preparation of compounds of this type, for example as shown in the following reaction Schemes. Unless otherwise indicated R¹ through R⁷ and A, V, W, X and Z in the reaction Schemes and discussion that follow are defined as above. The term “protecting group”, as used hereinafter, means a hydroxy or amino protecting group which is selected from typical hydroxy or amino protecting groups described in Protective Groups in Organic Synthesis edited by T. W. Greene et al. (John Wiley & Sons, 1991);

The following reaction Schemes illustrate the preparation of compounds of formula (I).

The compounds of the present invention may be prepared by a variety of processes well known for the preparation of compounds of this type, for example as shown in the following reaction Schemes. Unless otherwise indicated R¹ through R⁷ and A, V, W, X and Z in the reaction Schemes and discussion that follow are defined as above.

The following reaction Schemes illustrate the preparation of compounds of formula (I).

Scheme 1:

This illustrates the preparation of compounds of formula (Ib) wherein R² represents a hydroxy group.

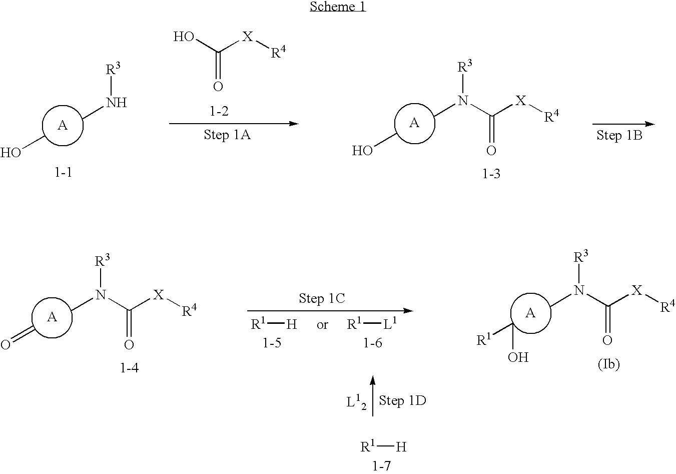

In the above formula, L¹ represents a halogen atom such as, chlorine, bromine or iodine.

Step 1A

In this Step, an amide compound of formula 1-3 can be prepared by the coupling reaction of an amine compound of formula 1-2 with an acid compound of formula 1-2 in the presence or absence of a coupling reagent in an inert solvent. If desired, this reaction may be carried out in the presence or absence of an additive such as 1-hydoroxybenzotriazole or 1-hydroxyazabenzotriazole.

The reaction is normally and preferably effected in the presence of a solvent. There is no particular restriction on the nature of the solvent to be employed, provided that it has no adverse effect on the reaction or on the reagents involved and that it can dissolve the reagents, at least to some extent. Examples of suitable solvents include: acetone, dimethylformamide, acetonitrile; halogenated hydrocarbons, such as dichloromethane, dichloroethane, chloroform; and ethers, such as tetrahydrofuran and dioxane.

The reaction can take place over a wide range of temperatures, and the precise reaction temperature is not critical to the invention. The preferred reaction temperature will depend upon such factors as the nature of the solvent, and the starting material or reagent used. However, in general, we find it convenient to carry out the reaction at a temperature of from −20° C. to 100° C., more preferably from about 0° C. to 60° C. The time required for the reaction may also vary widely, depending on many factors, notably the reaction temperature and the nature of the reagents and solvent employed. However, provided that the reaction is effected under the preferred conditions outlined above, a period of 5 minutes to 1 week, more preferably 30 minutes to 24 hours, will usually suffice.

Suitable coupling reagents are those typically used in peptide synthesis including, for example, diimides (e.g., dicyclohexylcarbodiimide (DCC), water soluble carbodiimide (WSC)), 2-ethoxy-N-ethoxycarbonyl-1,2-dihydroquinoline, benzotriazol-1-yloxy-tris(dimethylamino)phosphonium hexafluorophosphate (BOP), diethyl azodicarboxylate-triphenylphosphine, diethylcyanophosphate, diethylphosphorylazide, 2-chloro-1-methylpyridinium iodide, or ethyl chloroformate.

Step 1B

The following oxidation can be carried out in the presence of an oxidative agent in a reaction-inert solvent such as aqueous or non-aqueous organic solvents. Examples of suitable solvents include: tetrahydrofuran, dioxane, acetone, dimethylformamide, acetonitrile, halogenated hydrocarbons (e.g., dichloromethane, dichloroethane, chloroform). Suitable oxidative agents include, for example, Cr-reagents, such as pyridium chlorochlomate, chromium oxide, pyridium dichlromate; Ru-reagents, such as tetrapropylammonium perruthenate, ruthenium tetraoxide; dimethyl sulfoxide with an activator, such as oxalyl chloride, DCC, sulphortrioxide-pyridine; and dimethyl sulfide with an activator, such as chlorine, N-chlorosuccinimide.

The reaction can take place over a wide range of temperatures, and the precise reaction temperature is not critical to the invention. The preferred reaction temperature will depend upon such factors as the nature of the solvent, and the starting material or reagent used. However, in general, we find it convenient to carry out the reaction at a temperature of from −78° C. to 100° C., more preferably from about −60° C. to 60° C. The time required for the reaction may also vary widely, depending on many factors, notably the reaction temperature and the nature of the reagents and solvent employed. However, provided that the reaction is effected under the preferred conditions outlined above, a period of 1 minute to 24 hours, more preferably 30 minutes to 12 hours, will usually suffice.

Step 1C

In this Step, the compound of formula (Ib) can be prepared by the coupling reaction of a ketone compound of formula 1-4 with R¹—H compound of formula 1-5 or R¹-L¹ compound of formula 1-6 in the presence of a metallic reagent. If desired, this reaction may be carried out in the presence or absence of an additive, such as hexamethylphosphoramide (HMPA) tetramethylethylenediamine (TMEDA), or cerium dichloride, usually in excess.

The reaction is normally and preferably effected in the presence of a solvent. There is no particular restriction on the nature of the solvent to be employed, provided that it has no adverse effect on the reaction or on the reagents involved and that it can dissolve the reagents, at least to some extent. Examples of suitable solvents include: tetrahydrofuran, ether, toluene, ethyleneglycol dimethylether or dioxane.

The reaction can take place over a wide range of temperatures, and the precise reaction temperature is not critical to the invention. The preferred reaction temperature will depend upon such factors as the nature of the solvent, and the starting material or reagent used. However, in general, we find it convenient to carry out the reaction at a temperature of from −100° C. to 20° C., more preferably from about −78° C. to 0° C. The time required for the reaction may also vary widely, depending on many factors, notably the reaction temperature and the nature of the reagents and solvent employed. However, provided that the reaction is effected under the preferred conditions outlined above, a period of 5 minutes to 24 hours, more preferably 30 minutes to 3 hours, will usually suffice.

Suitable metallic reagents include; alkyl lithiums, such as n-butyllithium, sec-butyllithium or tert-butyllithium; aryllithiums, such as phenyllithium or lithium naphtilide; methalamide such as sodium amide or lithium diisopropylamide; and alkali-metal, such as potassium hydride, sodium hydride, Mg, Na, or Zn.

Step 1D

The halogenated compound 1-6 may be generally prepared by halogenation with a halogenating reagent in a reaction-inert solvent. Examples of suitable solvents include: such as aqueous or non-aqueous organic solvents such as tetrahydrofuran, dioxane, acetone, dimethylformamide, acetonitrile; halogenated hydrocarbons, such as dichloromethane, dichloroethane or chloroform; and acetic acid. Suitable halogenating reagents include, for example, bromine, chlorine, iodine, N-chlorosuccimide, N-bromosuccimide, 1,3-dibromo-5,5-dimethylhydantoin, bis(dimethylacetamide)hydrogen tribromide, tetrabutylammonium tribromide, bromodimethylsulfonium bromide, hydrogen bromide-hydrogen peroxide, nitrodibromoacetonitrile or copper(II) bromide. The reaction can take place over a wide range of temperatures, and the precise reaction temperature is not critical to the invention. The preferred reaction temperature will depend upon such factors as the nature of the solvent, and the starting material or reagent used. However, in general, we find it convenient to carry out the reaction at a temperature of from 0° C. to 200° C., more preferably from 20° C. to 120° C. The time required for the reaction may also vary widely, depending on many factors, notably the reaction temperature and the nature of the reagents and solvent employed. However, provided that the reaction is effected under the preferred conditions outlined above, a period of 5 minutes to 48 hours, more preferably 30 minutes to 24 hours, will usually suffice.

Scheme 2:

This illustrates the alternative preparation of compounds of formula (Ib) wherein R² represents a hydroxy group.

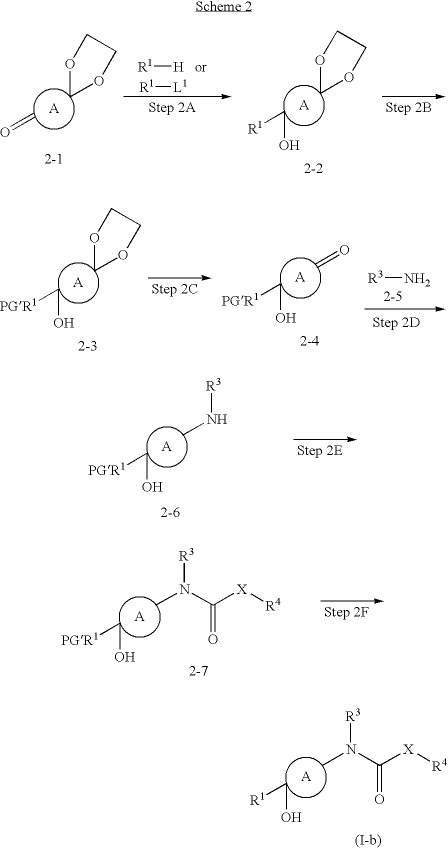

In the above formula, PG′ represents a protecting group. The term “protecting group”, as used herein, means a hydroxy or amino protecting group which is selected from typical hydroxy or amino protecting groups described in Protective Groups in Organic Synthesis edited by T. W. Greene et al. (John Wiley & Sons, 1991). Typical hydroxy or amino protecting groups include benzyl, C₂H₅O(C═O)—, CH₃(C═O)—, t-butyldimethylsilyl (TBS), t-butyldiphenylsilyl, benzyloxycarbonyl represented as Z and t-buthoxycarbonyl represented as t-Boc or Boc.

Step 2A

In this Step, an alcohol compound of formula 2-2 can be prepared by the coupling reaction of a ketone compound of formula 2-1 with R¹—H compound of formula 1-5 or R¹-L¹ compound of formula 1-6 in the presence of a metallic reagent in a reaction-inert solvent. If desired, this reaction may be carried out in the presence or absence of an additive, such as hexamethylphosphoramide (HMPA) tetramethylethylenediamine (TMEDA), or cerium trichloride, usually in excess.

The reaction is normally and preferably effected in the presence of a solvent. There is no particular restriction on the nature of the solvent to be employed, provided that it has no adverse effect on the reaction or on the reagents involved and that it can dissolve the reagents, at least to some extent. Examples of suitable solvents include: tetrahydrofuran, ether, toluene, ethyleneglycol dimethylether or dioxane.

The reaction can take place over a wide range of temperatures, and the precise reaction temperature is not critical to the invention. The preferred reaction temperature will depend upon such factors as the nature of the solvent, and the starting material or reagent used. However, in general, we find it convenient to carry out the reaction at a temperature of from −100° C. to 20° C., more preferably from about −78° C. to 0° C. The time required for the reaction may also vary widely, depending on many factors, notably the reaction temperature and the nature of the reagents and solvent employed. However, provided that the reaction is effected under the preferred conditions outlined above, a period of 5 minutes to 24 hours, more preferably 30 minutes to 3 hours, will usually suffice.

Suitable metallic reagents include; alkyl lithiums, such as n-butyllithium, sec-butyllithium or tert-butyllithium; aryllithiums, such as phenyllithium or lithium naphtilide; and alkali-metal, such as potassium hydride, sodium hydride, Mg, Na, or Zn.

Step 2B

In this Step, a protected compound of formula 2-3 can be prepared by the deprotonation of a hydroxy or an amino group of the compound of formula 2-2 with a metallic reagent followed by the introducing the protecting group defined above in a reaction-inert solvent.

The deprotonation is normally and preferably effected in the presence of a solvent. There is no particular restriction on the nature of the solvent to be employed, provided that it has no adverse effect on the reaction or on the reagents involved and that it can dissolve the reagents, at least to some extent. Examples of suitable solvents include: tetrahydrofuran, dimethylformamide, dimethylsulfoxide, ether, toluene, ethyleneglycol dimethylether generally or dioxane.

The deprotonation can take place over a wide range of temperatures, and the precise reaction temperature is not critical to the invention. The preferred reaction temperature will depend upon such factors as the nature of the solvent, and the starting material or reagent used. However, in general, we find it convenient to carry out the reaction at a temperature of from −50° C. to 70° C., more preferably from about 0° C. to 50° C. The time required for the reaction may also vary widely, depending on many factors, notably the reaction temperature and the nature of the reagents and solvent employed. However, provided that the reaction is effected under the preferred conditions outlined above, a period of 5 minutes to 12 hours, more preferably 30 minutes to 3 hours, will usually suffice.

Examples of suitable bases for deprotonation or proton scavenger include: alkyl lithiums, such as n-butyllithium, sec-butyllithium or tert-butyllithium; aryllithiums, such as phenyllithium or lithium naphtilide; and alkali metal, such as potassium hydride or sodium hydride; amines, such as triethylamine, pyridine, or imidazole.

Introducing the protecting group may be carried out by using, for example, appropriate benzylhalide, such as benzylbromide or benzylchloride; silyl halides; aralkyl halide; acid halides; acid anhydride and acids, such as benzyl, t-butyldimethylsilyl (TBS) chloride, t-butyldiphenylsilylchloride, Z-chloride and t-BocCl or Boc₂O, using the methods described in Protective Groups in Organic Synthesis edited by T. W. Greene et al. (John Wiley & Sons, 1991).

The reaction can take place over a wide range of temperatures, and the precise reaction temperature is not critical to the invention. The preferred reaction temperature will depend upon such factors as the nature of the solvent, and the starting material or reagent used. However, in general, we find it convenient to carry out the reaction at a temperature of from 0° C. to 120° C., more preferably from 0° C. to 70° C. The time required for the reaction may also vary widely, depending on many factors, notably the reaction temperature and the nature of the reagents and solvent employed. However, provided that the reaction may be effected under the preferred conditions outlined above, a period of from 5 minutes to 48 hours, more preferably from 30 minutes to 24 hours, will usually suffice.

Step 2C

In this Step, a ketone compound of formula 2-4 can be prepared by the hydrolysis reaction of a ketal compound of formula 2-3 in the presence or the absence of a catalyst in a reaction-inert solvent.

The hydrolysis reaction may be carried out in an aqueous or non-aqueous organic solvent. Examples of suitable solvents include: alcohols, such as methanol or ethanol; ethers, such as tetrahydrofuran or dioxane; acetone; dimethylformamide; halogenated hydrocarbons, such as dichloromethane, dichloroethane or chloroform; acids, such as acetic acid, hydrogen chloride, hydrogen bromide and sulfuric acid. Example of suitable catalysts include: hydrogen halides, such as hydrogen chloride and hydrogen bromide; sulfonic acids, such as p-toluenesulfonic acid and benzenesulfonic acid; ammonium salts, such as pyridium p-toluenesulfonate and ammonium chloride; and carboxylic acid, such as acetic acid and trifluoroacetic acid. This reaction can be carried out at temperature of 0° C. to 200° C., preferably from about 20° C. to 120° C. for 5 minutes to 48 hours, preferably 30 minutes to 24 hours.

Step 2D

In this Step, an amine compound of formula 2-6 can be prepared by the reductive amination of the ketone compound of formula 2-4 with an amine compound of formula 2-5 in the presence or absence of a reducing agent or a metal agent in an inert solvent.

The reaction is normally and preferably effected in the presence of a solvent. There is no particular restriction on the nature of the solvent to be employed, provided that it has no adverse effect on the reaction or on the reagents involved and that it can dissolve the reagents, at least to some extent. Examples of suitable aqueous or non-aqueous organic solvents include: alcohols, such as methanol, ethanol or isopropanol; ethers, such as tetrahydrofuran, dimethoxyethane or dioxane; acetone; acetonitrile; dimethylformamide, acetic acid; and halogenated hydrocarbon, such as dichloromethane, dichloroethane or chloroform.

The reaction can take place over a wide range of temperatures, and the precise reaction temperature is not critical to the invention. The preferred reaction temperature will depend upon such factors as the nature of the solvent, and the starting material or reagent used. However, in general, we find it convenient to carry out the reaction with reducing agents at a temperature of from −78° C. to 100° C., more preferably from about −20° C. to 60° C. The time required for the reaction may also vary widely, depending on many factors, notably the reaction temperature and the nature of the reagents and solvent employed. However, provided that the reaction is effected under the preferred conditions outlined above, a period of 5 minutes to 1 week, more preferably 30 minutes to 24 hours, will usually suffice. In the case of the reaction with metal reagents, we find it convenient to carry out the reaction at a temperature of from 20° C. to 100° C., preferably from about 20° C. to 60° C. for 10 minutes to 48 hours, preferably 30 minutes to 24 hours.

Suitable reducing reagents are those typically used in the reduction including, for example, sodium borohydride, sodium cyanoborohydride, sodium triacetoxyborohydride.

Example of suitable metal reagents include palladium-carbon, palladiumhydroxide-carbon, platinumoxide, platinum-carbon, ruthenium-carbon, rhodium-aluminumoxide and tris[triphenyphosphine] rhodiumchlrodie.

The reduction with metal reagents may be carried out under hydrogen atmosphere at a pressure ranging from 1 to 100 atom, preferably from 1 to 10 atom.

This reduction can be carried out after formation of the corresponding enamine of the compound 2-4 or imine of the compound of 2-4 in a reaction-inert solvent such as benzene, toluene, or xylene at a temperature in the range from 80 to 130° C. for 1 hour to 1 week.

Step 2E

In this Step, a desired amide compound of formula 2-7 may be prepared by coupling reaction of the amine compound of formula 2-6 with the acid compound of formula 1-2 described in Scheme 1.

This reaction is essentially the same as and may be carried out in the same manner as and using the same reagents and reaction conditions as Step 1A in Scheme 1.

Step 2F

In this Step, the desired compound of formula (Ib) may be prepared by the deprotection of the compound of formula 2-7, prepared as described in Step 2E, according to known procedures such as those described in Protective Groups in Organic Synthesis edited by T. W. Greene et al. (John Wiley & Sons, 1991).

In the case of Bn or Z protection, the removal of the protecting groups may be carried out under, for example, known hydrogenolysis conditions in the presence of a metal catalyst under hydrogen atmosphere or in the presence of hydrogen sources such as formic acid or ammonium formate in a reaction inert solvent. If desired, the reaction is carried out under acidic conditions, for example, in the presence of hydrochloric acid or acetic acid. A preferred metal catalyst is selected from, for example, palladium-carbon, palladiumhydroxide-carbon, platinumoxide, platinum-carbon, ruthenium-carbon, rhodium-aluminumoxide, tris[triphenyphosphine] rhodiumchlrodie. Example of suitable reaction inert aqueous or non-aqueous organic solvents include: alcohols, such as methanol, ethanol; ethers, such as tetrahydrofuran or dioxane; acetone; dimethylformamide; halogenated hydrocarbons, such as dichloromethane, dichloroethane or chloroform; and acetic acid or mixtures thereof. The reaction may be carried out at a temperature in the range from of 20° C. to 100° C., preferably in the range of 20° C. to 60° C. Reaction times are, in general, from 10 minutes to 48 hours, preferably 30 minutes to 24 hours. This reaction may be carried out under hydrogen atmosphere at a pressure ranging from 1 to 100 atom, preferably from 1 to 10 atom.

Scheme 3:

This illustrates a preparation of compounds of formula (Ic) wherein R² represents a hydrogen atom.

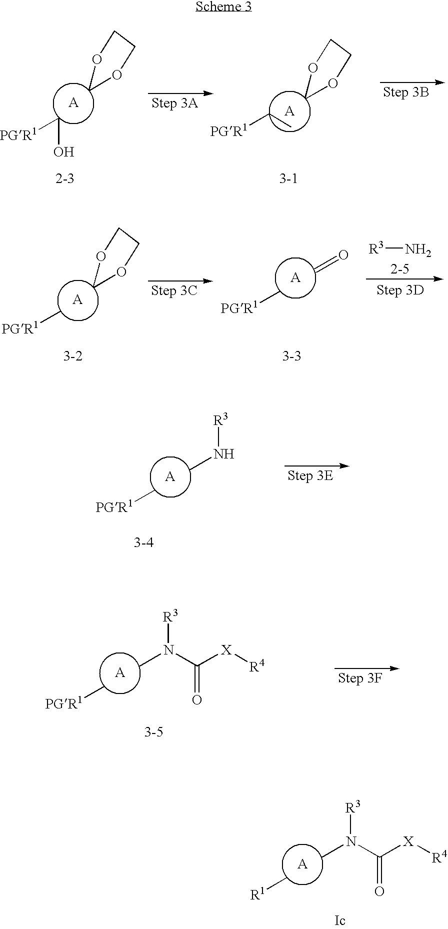

Step 3A

In this Step, an olefin compound of formula 3-1 can be prepared by the dehydration reaction of the alcohol compound of formula 2-3, which can be prepared by the method described in Step 2B in Scheme 2, in the presence of a dehydrating agent in the presence or absence of an appropriate base in a reaction-inert solvent. If desired, this reaction may be carried out in the presence or absence of a base.

Example of suitable solvents include: aromatic hydrocarbons, such as benzene, toluene and xylene; alcohols, such as methanol, ethanol, propanol and isopropanol; ethers, such as tetrahydrofuran and dioxane; acetone; dimethylformamide; halogenated hydrocarbons, such as dichloromethane, dichloroethane and chloroform; and acetic acid. Example of a suitable dehydrating agents include: hydrogen halide, such as hydrogen chloride and hydrogen bromide; sulfonic acids, such as p-toluenesulfonic acid and benzenesulfonic acid; sulfonylchloride, such as metansulfonylchloride and p-toluenesulfonylchloride; methoxycarbonylsulfamoyl)triethylammonium hydroxide; and p-toluenesulfonylisocyanate. Example of suitable bases include: alkylamines, such as triethylamine and diisopropylethylamine; aromatic amines, such as pyridine and imidazole; and inorganic bases, such as potassium carbonate and sodium hydroxide. This reaction can be carried out at temperature of 0° C. to 200° C., preferably from about ambient temperature to 120° C. for 5 minutes to 48 hours, preferably 30 minutes to 24 hours.

Step 3B

In this Step, a desired dehydroxy compound of formula 3-2 may be prepared by reduction of the olefin compound of formula 3-1 with the reducing agent.

This reaction is essentially the same as and may be carried out in the same manner as and using the same reagents and reaction conditions as Step 2F in Scheme 2.

Step 3C

In this Step, a desired ketone compound of formula 3-3 may be prepared by the hydrolysis of the ketal compound of formula 3-2.

This reaction is essentially the same as and may be carried out in the same manner as and using the same reagents and reaction conditions as Step 2C in Scheme 2.

Step 3D

In this Step, a desired amine compound of formula 3-4 may be prepared by the reductive amination of the ketone compound of formula 3-3 with an amine compound of formula 2-5.

This reaction is essentially the same as and may be carried out in the same manner as and using the same reagents and reaction conditions as Step 2D in Scheme 2.

Step 3E

In this Step, a desired amide compound of formula 3-5 may be prepared by coupling reaction of the amine compound of formula 3-4 with the acid compound of formula 1-2 described in Scheme 1.

This reaction is essentially the same as and may be carried out in the same manner as and using the same reagents and reaction conditions as Step 1A in Scheme 1.

Step 3F

In this Step, the desired compound of formula Ic may be prepared by the deprotection of the compound of formula 3-5, prepared as described in Step 3E.

This reaction is essentially the same as and may be carried out in the same manner as and using the same reagents and reaction conditions as Step 2F in Scheme 2.

Scheme 4:

This illustrates a preparation of compounds of formula (Id) wherein R¹⁰ represents an alkyl groups having from 1 to 6 carbon atoms.

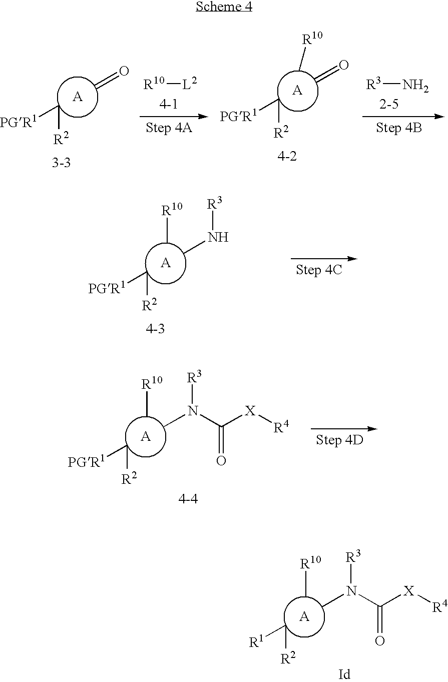

In the above formula, R¹⁰ represents an alkyl groups having from 1 to 6 carbon atoms; and L² represents a halogen atom such as, chlorine, bromine or iodine.

Step 4A

In this Step, an alkyl compound of formula 4-2 can be prepared by the deprotonation followed by the alkylation of the ketone compound of formula 3-3 with a metallic reagent and an alkylating agent in a reaction-inert solvent.

The deprotonation is normally and preferably effected in the presence of a solvent. There is no particular restriction on the nature of the solvent to be employed, provided that it has no adverse effect on the reaction or on the reagents involved and that it can dissolve the reagents, at least to some extent. Examples of suitable solvents include: tetrahydrofuran, dimethylformamide, dimethylsulfoxide, ether, toluene, ethyleneglycol dimethylethergenerally or dioxane.

The deprotonation can take place over a wide range of temperatures, and the precise reaction temperature is not critical to the invention. The preferred reaction temperature will depend upon such factors as the nature of the solvent, and the starting material or reagent used. However, in general, we find it convenient to carry out the reaction at a temperature of from −50° C. to 70° C., more preferably from about 0° C. to 50° C. The time required for the reaction may also vary widely, depending on many factors, notably the reaction temperature and the nature of the reagents and solvent employed. However, provided that the reaction is effected under the preferred conditions outlined above, a period of 5 minutes to 12 hours, more preferably 30 minutes to 3 hours, will usually suffice.

Examples of suitable metallic reagents include: for example, alkyl lithiums, such as n-butyllithium, sec-butyllithium or tert-butyllithium; aryllithiums, such as phenyllithium or lithium naphtilide; methalamide such as sodium amide or lithium diisopropylamide; and alkali metal, such as potassium hydride or sodium hydride.

The alkylation may be carried out by using, for example, appropriate alkylhalide, such as methyliodide and ethyliodide.

The reaction can take place over a wide range of temperatures, and the precise reaction temperature is not critical to the invention. The preferred reaction temperature will depend upon such factors as the nature of the solvent, and the starting material or reagent used. However, in general, we find it convenient to carry out the reaction at a temperature of from 0° C. to 120° C., more preferably from 0° C. to 70° C. The time required for the reaction may also vary widely, depending on many factors, notably the reaction temperature and the nature of the reagents and solvent employed. However, provided that the reaction may be effected under the preferred conditions outlined above, a period of from 5 minutes to 48 hours, more preferably from 30 minutes to 24 hours, will usually suffice.

Step 4B, 4C and 4D

In these Steps, the desired compound of formula (Id) may be prepared by the reductive amination, the coupling reaction and deprotection. These reactions are essentially the same as and may be carried out in the same manner as and using the same reagents and reaction conditions as Step 3D, 3E and 3F in Scheme 3.

Scheme 5:

This illustrates an alternative preparation of compounds of formula (Ib) wherein R² represents a hydroxy group.

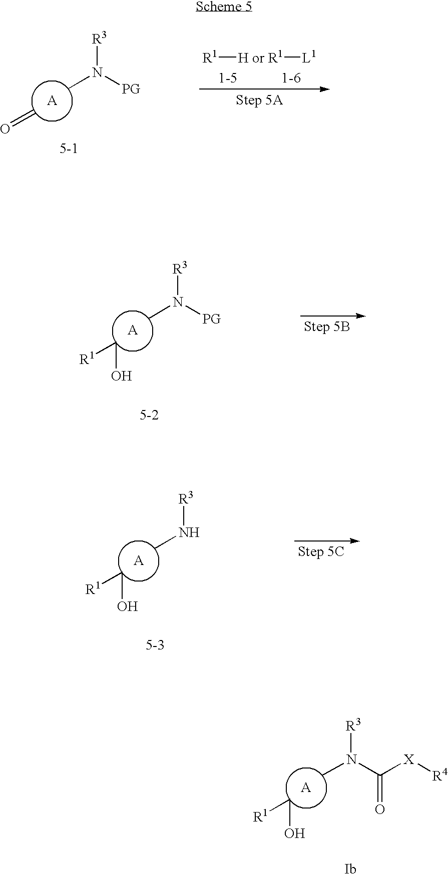

In the above formula, L¹ represents a halogen atom such as, chlorine, bromine or iodine; and PG represents a protecting group. The term “protecting group”, as used herein, means amino protecting group which is selected from typical amino protecting groups described in Protective Groups in Organic Synthesis edited by T. W. Greene et al. (John Wiley & Sons, 1991). Typical amino protecting groups include benzyl, C₂H₅O(C═O)—, CH₃(C═O)—, t-butyldimethylsilyl (TBS), t-butyldiphenylsilyl, benzyloxycarbonyl represented as Z and t-buthoxycarbonyl represented as t-Boc or Boc.

Step 5A

In this Step, an alcohol compound of formula 5-2 may be prepared the coupling reaction of a ketone compound of formula 5-1, which may be prepared by the known method described in EP366059, with R¹—H compound of formula 1-5 or R¹-L¹ compound of formula 1-6 in the presence of a metallic agent. This reaction is essentially the same as and may be carried out in the same manner as and using the same reagents and reaction conditions as Step 1C in Scheme 1.

Step 5B

In this Step, an amine compound of formula 5-3 may be prepared the deprotection of the compound of formula 5-2. This reaction is essentially the same as and may be carried out in the same manner as and using the same reagents and reaction conditions as Step 2F in Scheme 2.

Step 5C

In this Step, the desired amide compound of formula Ib may be prepared by coupling reaction of the amine compound of formula 5-3 with the acid compound of formula 1-2 described in Scheme 1.

This reaction is essentially the same as and may be carried out in the same manner as and using the same reagents and reaction conditions as Step 1A in Scheme 1.

Scheme 6:

This illustrates a preparation of compounds of formula (Ie) wherein A′ represents a heterocyclic group having from 4 to 10 atoms which consists of at least one carbon atom and nitrogen atom, and from 1 to 4 heteroatoms selected from the atoms consisting of sulfur atoms, oxygen atoms and nitrogen atoms.

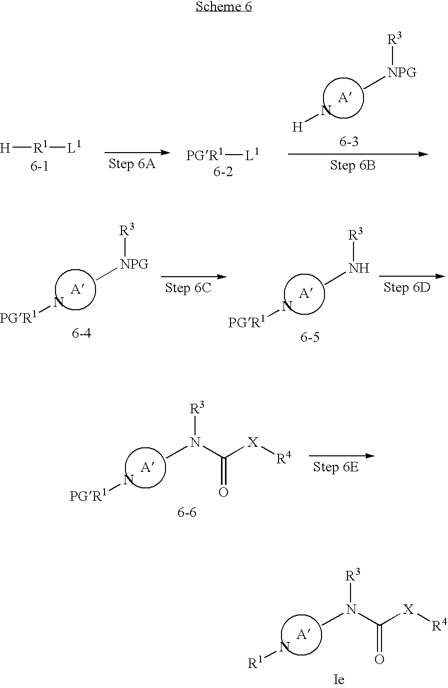

In the above formula, A′ represents a heterocyclic group having from 4 to 10 atoms which consists of at least one carbon atom and nitrogen atom, and from 1 to 4 heteroatoms selected from the atoms consisting of sulfur atoms, oxygen atoms and nitrogen atoms.

Step 6A

In this Step, a protected compound of formula 6-2 can be prepared by the deprotonation of a hydroxy or an amino group of the compound of formula 6-1 with a metallic reagent followed by the introducing the protecting group defined above in a reaction-inert solvent.

This reaction is essentially the same as and may be carried out in the same manner as and using the same reagents and reaction conditions as Step 2B in Scheme 2.

Step 6B

In this Step, a desired compound of formula 6-4 can be prepared by the coupling reaction of the protected compound of formula 6-2 with an amine compound of formula 6-3 in the presence or absence of catalyst and/or a base in an inert solvent. If desired, this reaction may be carried out in the presence or absence of a ligand such as, triphenylphosphine.

The reaction is normally and preferably effected in the presence of a solvent. There is no particular restriction on the nature of the solvent to be employed, provided that it has no adverse effect on the reaction or on the reagents involved and that it can dissolve the reagents, at least to some extent. Examples of aqueous or non-aqueous organic solvents include: alcohols, such as methanol and ethanol; ethers, such as tetrahydrofuran and dioxane; acetone; dimethylformamide; acetonitrile; halogenated hydrocarbons, such as dichloromethane, dichloroethane and chloroform; and aromatic hydrocarbons, such as toluene, benzene and xylene.

The reaction can take place over a wide range of temperatures, and the precise reaction temperature is not critical to the invention. The preferred reaction temperature will depend upon such factors as the nature of the solvent, and the starting material or reagent used. However, in general, we find it convenient to carry out the reaction at a temperature of from 0° C. to 300° C., more preferably from about 20° C. to 150° C. The time required for the reaction may also vary widely, depending on many factors, notably the reaction temperature and the nature of the reagents and solvent employed. However, provided that the reaction is effected under the preferred conditions outlined above, a period of 5 minutes to 1 week, more preferably 30 minutes to 24 hours, will usually suffice.

Example of suitable catalysts include: palladium reagents, such as palladium acetate and palladium dibenzylacetone; and copper reagents, such as copper acetate and copper. Example of suitable bases include: potassium carbonate, sodium tert-butoxide, sodium hydride and potassium hydride.

Step 6C

In this Step, a desired compound of formula 6-5 may be prepared by the deprotection of the compound of formula 6-4, according to known procedures such as those described in Protective Groups in Organic Synthesis edited by T. W. Greene et al. (John Wiley & Sons, 1991).

In the case of Boc protection, the removal of the protecting groups may be carried out under known conditions in the presence or the absence of catalytic amount of an acid in a reaction inert solvent. Example of suitable aqueous or non-aqueous organic reaction inert solvents include: ethyl acetate; alcohols, such as methanol and ethanol; ethers, such as tetrahydrofuran and dioxane; acetone; dimethylformamide; halogenated hydrocarbons, such as dichloromethane, dichloroethane or chloroform; and acetic acid or mixtures thereof. The reaction may be carried out at a temperature in the range from of 0° C. to 200° C., preferably in the range of 20° C. to 120° C. Reaction times are, in general, from 5 minutes to 48 hours, preferably 30 minutes to 24 hours. Example of suitable catalysts include: hydrogen halide, such as hydrogen chloride and hydrogen bromide; sulfonic acids, such as p-toluenesulfonic acid and, benzenesulfonic acid; ammonium salts, such as pyridium p-toluenesulfonate and ammonium chloride; and carboxylic acid, such as acetic acid and trifluoroacetic acid.

Step 6D and 6E

In these Steps, a desired compound of formula Ie may be prepared by coupling reaction followed by the deprotection.

These reactions are essentially the same as and may be carried out in the same manner as and using the same reagents and reaction conditions as Step 1A in Scheme 1 and Step 2F in Scheme 2.

Scheme 7:

This illustrates a preparation of compounds of formula (If) wherein R² represents a hydroxy group and R³ represents a hydrogen atom.

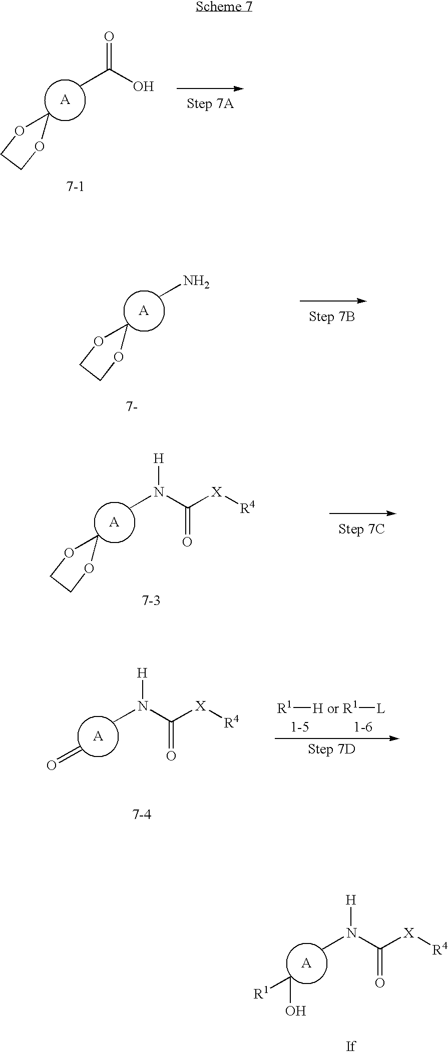

Step 7A

In this Step, an amine compound of formula 7-2 can be prepared by the rearrangement reaction of a carboxylic acid compound of formula 7-1, which may be prepared by the known procedure described in WO02/30890, in multi-step reactions including acylazide formation, rearrangement by heating, and hydrolysis of resulting isocyanate.

Acylazide formation can be carried out using an azide reagent in the presence or absence of a coupling agent in a reaction-inert solvent. Example of suitable solvents include: ethers, such as tetrahydrofuran, ethyleneglycol dimethylether, dioxane and diethyl ethers; dimethylformamide; dimethylsulfoxide; and toluene. Example of suitable azide reagents include sodium azide and diethylphosphoryl azide. Example of suitable coupling agents include: diimides, such as dicyclohexylcarbodiimide (DCC), water soluble carbodiimide (WSC)), 2-ethoxy-N-ethoxycarbonyl-1,2-dihydroquinoline, benzotriazol-1-yloxy-tris(dimethylamino)phosphonium hexafluorophosphate (BOP), diethyl azodicarboxylate-triphenylphosphine, diethylcyanophosphate, diethylphosphorylazide, and ethyl chloroformate. If desired this reaction may be carried out in the presence or the absence of an additive such as 1-hydoroxybenzotriazole or 1-hydroxyazabenzotriazole. This reaction can be carried out at a temperature in the range from −20° C. to 100° C., preferably from about 0° C. to 60° C. for 5 minutes to 1 week, preferably 30 minutes to 24 hours. An acylazide can be formed via an acylhalide, which can be obtained by the reaction with halogenating agents such as oxalylchloride and thionyl chloride. The resulting acylazide can be converted to the corresponding isocyanate by heating at a temperature in the range from about 50° C. to 200° C., preferably from about 80° C. to 150° C. for 5 minutes to 1 week, preferably 30 minutes to 24 hours. The hydrolysis of isocyanates can be carried out using aqueous alkaline solutions such as sodium hydroxide and potassium hydroxide.

Step 7B, 7C and 7D

In these Steps, a desired compound of formula If wherein R² represents a hydroxy group and R³ represents a hydrogen atom, may be prepared by reactions consisting of the coupling reaction, deprotection and the additional coupling reaction.

These reactions are essentially the same as and may be carried out in the same manner as and using the same reagents and reaction conditions as Step 1A in Scheme 1, Step 2C in Scheme 2 and Step 1C in Scheme 1.

Scheme 8:

This illustrates a preparation of compounds of formula 8-3.

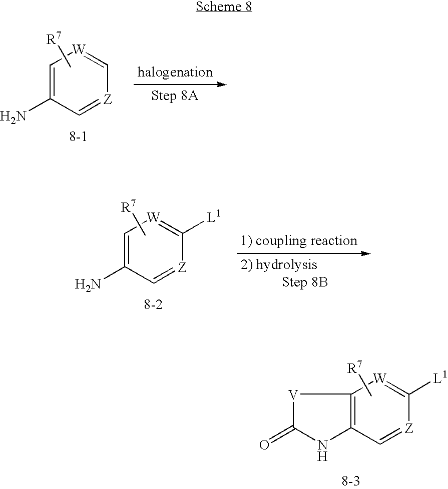

Step 8A

In this Step, a halogenated compound of formula 8-2 can be prepared by the halogenation of the compound of formula 8-1 with a halogenating reagent.

This reaction is essentially the same as and may be carried out in the same manner as and using the same reagents and reaction conditions as Step 1D in Scheme 1.

Step 8B

In this Step, a bicyclic compound of formula 8-3 may be prepared by coupling reaction followed by hydrolysis.

The coupling reaction with methyl malonate can be carried out in a reaction-inert solvent. Example of suitable aqueous or non-aqueous organic solvents include: alcohols, such as methanol and ethanol; ethers, such as tetrahydrofuran and dioxane; acetone; dimethylformamide; acetonitrile; halogenated hydrocarbons, such as dichloromethane, dichloroethane and chloroform; aromatic hydrocarbons, such as toluene, benzene and xylene. This reaction may be carried out in the presence or the absence of a catalyst and/or base at a temperature in the range from 0° C. to 200° C., preferably from about 20° C. to 120° C. for 5 minutes to 48 hours, preferably 30 minutes to 24 hours. Example of suitable catalysts include: palladium reagents, such as palladium acetate and palladium dibenzylacetone. If desired this reaction can be carried out in the presence or the absence of ligands, such as triphenylphosphine.

Hydrolytic decarboxylation can be carried out in a reaction inert solvent. Example of suitable solvents include: alcohols, such as methanol and ethanol; ethers tetrahydrofuran and dioxane; dimethylformamide. The solvents contain an aqueous alkaline solution such as sodium hydroxide, potassium hydroxide and potassium carbonate. This reaction can be carried out at a temperature in the range from 0° C. to 100° C., preferably from about 20° C. to 80° C. for 5 minutes to 48 hours, preferably 30 minutes to 24 hours. Then the reaction mixture can be acidified with an acid. Example of suitable acids include: hydrogen halide, such as hydrogen chloride and hydrogen bromide; sulfonic acids, such as p-toluenesulfonic acid and benzenesulfonic acid; ammonium salts, such as pyridium p-toluenesulfonate and ammonium chloride; carboxylic acids, such as acetic acid and trifluoroacetic acid. This reaction can be carried out at a temperature in the range from 0° C. to 200° C., preferably from about 20° C. to 120° C. for 5 minutes to 48 hours, preferably 30 minutes to 24 hours.

The bicyclic compound of formula 8-3 can be obtained by conventional methods known to those skilled in the art described in Chemistry of Heterocyclic Compounds, 1999, 35(2), 146-160; J. Chem. Soc., B, Phys. Org. 1966, (4), 285-91; and EP 296455.

The compound 8-3 is equivalent to R¹-L¹ in the previous schemes for the preparation of compounds of the invention.

The starting materials in the aforementioned general syntheses may be commercially available or obtained by conventional methods known to those skilled in the art.

In the above Schemes from 1 to 8, examples of suitable solvents include a mixture of any two or more of those solvents described in each Step.

The compounds of formula (I), and the intermediates above-mentioned preparation methods can be isolated and purified by conventional procedures, such as distillation, recrystallization or chromatographic purification.

The optically active compounds of this invention can be prepared by several methods. For example, the optically active compounds of this invention may be obtained by chromatographic separation, enzymatic resolution or fractional crystallization from the final compounds.

Several cycloalkylene amide compounds of this invention possess an asymmetric center. Hence, the compounds can exist in separated (+)- and (−)-optically active forms, as well as in racemic one thereof. The present invention includes all such forms within its scope. Individual isomers can be obtained by known methods, such as optically selective reaction or chromatographic separation in the preparation of the final product or its intermediate.

The subject invention also includes isotopically-labelled compounds, which are identical to those recited in formula (I), but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively. Compounds of the present invention, prodrugs thereof, pharmaceutically acceptable esters of said compounds and pharmaceutically acceptable salts of said compounds, of said esters or of said prodrugs which contain the aforementioned isotopes and/or other isotopes of other atoms are within the scope of this invention. Certain isotopically-labelled compounds of the present invention, for example those into which radioactive isotopes such as ³H and ¹⁴C are incorporated, are useful in drug and/or substrate tissue distribution assay. Tritiated, i.e., ³H, and carbon-14, i.e., ¹⁴C, isotopes are particularly preferred for their ease of presentation and detectability. Further, substitution with heavier isotopes such as deuterium, i.e., ²H, can afford therapeutic advantage resulting from greater metabolic stability, for example increased in vivo half-life or reduced dosage requirement and, hence, may be preferred in some circumstances. Isotopically labeled compounds of formula (I) of this invention and prodrugs thereof can generally be prepared by carrying out the procedure disclosed in above-disclosed Schemes and/or Examples and Preparations below, by submitting a readily available isotopically labeled reagent for a non-isotopically labeled reagent.

The present invention includes salt forms of the compounds (I) as obtained.

Certain compounds of the present invention are capable of forming pharmaceutically acceptable non-toxic cations. Pharmaceutically acceptable non-toxic cations of compounds of formula (I) may be prepared by conventional techniques by, for example, contacting said compound with a stoichiometric amount of an appropriate alkali or alkaline earth metal (sodium, potassium, calcium and magnesium) hydroxide or alkoxide in water or an appropriate organic solvent such as ethanol, isopropanol, mixtures thereof, or the like.

The bases which are used to prepare the pharmaceutically acceptable base addition salts of the acidic compounds of this invention of formula (I) are those which form non-toxic base addition salts, i.e., salts containing pharmaceutically acceptable cations, such as adenine, arginine, cytosine, lysine, benethamine (i.e., N-benzyl-2-phenyletylamine), benzathine (i.e., N,N-dibenzylethylenediamine), choline, diolamine (i.e., diethanolamine), ethylenediamine, glucosamine, glycine, guanidine, guanine, meglumine (i.e., N-methylglucamine), nicotinamide, olamine (i.e., ethanolamine), ornithine, procaine, proline, pyridoxine, serine, tyrosine, valine and tromethamine (i.e., tris or tris(hydroxymethyl)aminomethane). The base addition salts can be prepared by conventional procedures.

Insofar as the certain compounds of this invention are basic compounds, they are capable of forming a wide variety of different salts with various inorganic and organic acids.

The acids which are used to prepare the pharmaceutically acceptable acid addition salts of the basic compounds of this invention of formula (I) are those which form non-toxic acid addition salts, i.e., salts containing pharmaceutically acceptable anions, such as the chloride, bromide, iodide, nitrate, sulfate or bisulfate, phosphate or acid phosphate, acetate, lactate, citrate or acid citrate, tartrate or bi-tartrate, succinate, malate, fumarate, gluconate, saccharate, benzoate, methanesulfonate, ethanesulfonate, benzenesulfonate, p-toluenesulfonate, adipate, aspartate camsylate, (i.e., 1,2-ethanedisulfontate), estolate (i.e., laurylsulfate), gluceptate (i.e., gluscoheptonate), gluconate, 3-hydroxy-2-naphthoate, xionofoate (i.e., 1-hydrroxy-2-naphthoate), isethionate,(i.e., 2-hydroxyethanesulfonate), mucate (i.e., galactarate), 2-naphsylate (i.e., naphthalenesulphonate, stearate, cholate, glucuronate, glutamate, hippurate, lactobionate, lysinate, maleate, mandelate, napadisylate, nicatinate, polygalacturonate, salicylate, sulphosalicylate, tannate, tryptophanate, borate, carbonate, oleate, phthalate and pamoate (i.e., 1.1′-methylene-bis-(2-hydroxy-3-naphthoate). The acid addition salts can be prepared by conventional procedures.

For a review of on suitable salts see Berge et al., J. Pharm. Sci., 66, 1-19, 1977.

Also included within the scope of this invention are bioprecursors (also called pro-drugs) of the compounds of the formula (I). A bioprecursor of a compound of the formula (I) is a chemical derivative thereof which is readily converted back into the parent compound of the formula (I) in biological systems. In particular, a bioprecursor of a compound of the formula (I) is converted back to the parent compound of the formula (I) after the bioprecursor has been administered to, and absorbed by, a mammalian subject, e.g., a human subject. For example, it is possible to make a bioprecursor of the compounds of formula (I) in which one or both of L and W include hydroxy groups by making an ester of the hydroxy group. When only one of L and W includes hydroxy group, only mono-ester is possible. When both L and W include hydroxy, mono- and di-esters (which can be the same or different) can be made. Typical esters are simple alkanoate esters, such as acetate, propionate, butyrate, etc. In addition, when L or W includes a hydroxy group, bioprecursors can be made by converting the hydroxy group to an acyloxymethyl derivative (e.g., a pivaloyloxymethyl derivative) by reaction with an acyloxymethyl halide (e.g., pivaloyloxymethyl chloride).

When the compounds of the formula (I) of this invention may form solvates such as hydrates, such solvates are included within the scope of this invention.

### Method for Assessing Biological Activities

NR2B Binding Assay

The activity of the cycloalkylene amide compounds of the present invention, as NR2B antagonists, is determined by their ability to inhibit the binding of NR2B subunit at its receptor sites employing radioactive ligands.

The NR2B antagonist activity of the cycloalkylene amide compounds is evaluated by using the standard assay procedure described in, for example, J. Pharmacol., 331, pp117-126, 1997. This method essentially involves determining the concentration of the individual compound required to reduce the amount of radiolabelled NR2B ligands by 50% at their receptor sites, thereby affording characteristic IC₅₀ values for each compound tested. More specifically, the assay is carried out as follows.

Membranes were prepared by homogenization of forebrain of male CD rats weighing between 170˜190 g by using glass-Teflon homogenizer in 0.32 M sucrose at 4° C. The crude nuclear pellet was removed by centrifugation at 1000×g for 10 min, and the supernatant centrifuged at 17000×g for 25 min. The resulting pellet was resuspended in 5 mM Tris acetate pH 7.4 at 4° C. for 10 min to lyse cellular particles and again centrifuged at 17000×g. The resulting pellet (P2 membrane) was washed twice in Tris acetate, resuspended at 5.5 mg protein/ml and stored at −20° C. until use. All the manipulation was done on ice, and stock solution and equipment were kept on ice at all time.

For the saturation assay, receptor saturation was determined by incubating [³H]-CP-98,113 and 50 μg protein of P2 membrane for 60 minutes at room temperature in a final 100 μl of incubation buffer (50 mM Tris HCl, pH7.4). Total and non-specific bindings (in the presence of 10 μM of unlabeled CP-98,113) were determined in a range of [³H]-CP-98113 concentrations (0.625 nM to 60 nM). [³H]-CP-98,113 is as follows:

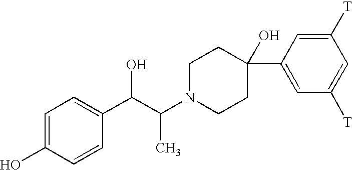

(wherein T is tritio (3H)).

For the competition assay, test compounds were incubated in duplicate with 5 nM [³H]-CP-98,113 and 50 μg protein of P2 membrane for 60 minutes at room temperature in a final 100 μl of 50 mM Tris HCl buffer (pH7.4). Nonspecific binding was determined by 10 μM of unlabeled CP-98,113 (25 μl). The saturation derived K_{D} gained in saturation assay was used for all Ki calculations.

All incubations were terminated by rapid vacuum filtration over 0.2% polyethyleneimine soaked Whatman GF/B glass fibre filter paper using a SKATRON cell harvester followed by three washes with ice-cold filtration buffer (5 mM Tris HCl, pH 7.4.). Receptor-bound radioactivity was quantified by liquid scintillation counting using Packard LS counter. Competition assays were performed by counting Wallac GF/B filters on Betaplate scintillation counter (Wallac).

Preferred compounds in this invention were tested by this method, and they showed Ki values from 2.7 nM to 8.9 nM with respect to inhibition of binding at the NR2B receptor.

Human NR2B Cell Functional Assay

HEK293 cells stably expressing human NR1b/2B receptor were used for cell functional assay. Cells were grown in 75-cm² culture flasks, using Dulbecco's modified Eagle's medium (DMEM, high glucose) supplemented with 10% fetal bovine, 52 μg/ml Zeocin, 530 μg/ml Geneticin, 100 units/ml penicillin and 100 μg/ml streptomycin. Cells were maintained in a humidified atmosphere in 5% CO₂ at 37° C., and 50-60% confluent cells were harvested by 0.05% trypsin containing 0.53 mM EDTA. The day before the experiment, expression of NR1b/2B receptor was induced by 5 μM ponasteron A in DMEM (40 ml) in the presence of 400 μM ketamine to prevent excitotoxicity. The induction was performed for 19-24 hours, using 50-60% confluent cells.

Cells were washed with 10 ml of Ca²⁺-free Krebs-Ringer Hepes buffer (KRH) containing 400 μM ketamine, and the loading of 5 μM fura-2 acetoxymethyl ester was made for 2 hrs at room temperature in the presence of 400 μM ketamine in Ca²⁺-free KRH (10 ml). Subsequently, cells were collected in 50 ml tube by pipetting manipulation and centrifuged at 850 rpm for 2 min. Supernatant was removed, and cells were washed with 10 ml of Ca²⁺-free KRH buffer, followed by centrifugation again. This manipulation was repeated 4 times to remove ketamine, glutamate and glycine. Cells were re-suspended in Ca²⁺-free KRH buffer, and 50 μl of cell suspension was added to each well of 96-well plates at a density of 100,000 cells/well, followed by adding test compounds dissolved in 50 μl of Ca²⁺-free KRH. After pre-incubation for 30 min, agonists (final 100 μM glutamic acid and 10 μM glycine) dissolved in 25 μl of KRH containing 9 mM Ca²⁺ (final 1.8 mM) were added. Fura-2 fluorescence (excitation wavelengths: 340 nm and 380 nm; emission wavelengths 510-520 nm) was monitored with a fluorescence imaging system, FDSS6000. The Δ fluorescence ratio F340/F380 (i.e., the fluorescence ratio immediately post-agonist—the basal fluorescence ratio; calculated as AUC) was used for evaluation of drug effects on agonists-induced changes in intracellular Ca²⁺. The basal fluorescence ratio was determined in the presence of 10 μM MK-801.

Rat Haloperidol-Induced Catalepsy Assay:

Fasted male CD rats were used (7-8 weeks old). Test compound or vehicle was given subcutaneously then haloperidol 0.5 mg/kg s.c. Sixty minutes after haloperidol-injection, the duration of catalepsy was quantified by placing the animals forepaws on an elevated bar and determining the latency to remove both forepaws from the bar. The cutoff latency was 60 seconds. Experimenter was blind to treatments during testing.

Human Dofetilide Binding

Human HERG transfected HEK293S cells were prepared and grown in-house. The collected cells were suspended in 50 mM Tris-HCl (pH 7.4 at 4° C.) and homogenized using a hand held Polytron PT 1200 disruptor set at full power for 20 sec on ice. The homogenates were centrifuged at 48,000×g at 4° C. for 20 min. The pellets were then resuspended, homogenized, and centrifuged once more in the same manner. The final pellets were resuspended in an appropriate volume of 50 mM Tris-HCl, 10 mM KCl, 1 mM MgCl₂ (pH 7.4 at 4° C.), homogenized, aliquoted and stored at −80° C. until use. An aliquot of membrane fractions was used for protein concentration determination using BCA protein assay kit (PIERCE) and ARVOsx plate reader (Wallac).

Binding assays were conducted in a total volume of 200 μl in 96-well plates. Twenty μl of test compounds were incubated with 20 μl of [³H]-dofetilide (Amersham, final 5 nM) and 160 μl of membrane homogenate (25 μg protein) for 60 minutes at room temperature. Nonspecific binding was determined by 10 μM dofetilide at the final concentration. Incubation was terminated by rapid vacuum filtration over 0.5% presoaked GF/B Betaplate filter using Skatron cell harvester with 50 mM Tris-HCl, 10 mM KCl, 1 mM MgCl₂, pH 7.4 at 4° C. The filters were dried, put into sample bags and filled with Betaplate Scint. Radioactivity bound to filter was counted with Wallac Betaplate counter.

I_{HERG} Assay

HEK 293 cells which stably express the HERG potassium channel were used for electrophysiological study. The methodology for stable transfection of this channel in HEK cells can be found elsewhere (Z. Zhou et al., 1998, Biophysical journal, 74, pp230-241). Before the day of experimentation, the cells were harvested from culture flasks and plated onto glass coverslips in a standard MEM medium with 10% FCS. The plated cells were stored in an incubator at 37° C. maintained in an atmosphere of 95%02/5% CO₂. Cells were studied between 15-28 hrs after harvest.

HERG currents were studied using standard patch clamp techniques in the whole-cell mode. During the experiment the cells were superfused with a standard external solution of the following composition (mM); NaCl, 130; KCl, 4; CaCl₂, 2; MgCl₂, 1; Glucose, 10; HEPES, 5; pH 7.4 with NaOH. Whole-cell recordings was made using a patch clamp amplifier and patch pipettes which have a resistance of 1-3 MOhm when filled with the standard internal solution of the following composition (mM); KCl, 130; MgATP, 5; MgCl₂, 1.0; HEPES, 10; EGTA 5, pH 7.2 with KOH. Only those cells with access resistances below 15 MΩ and seal resistances >1 GΩ was accepted for further experimentation. Series resistance compensation was applied up to a maximum of 80%. No leak subtraction was done. However, acceptable access resistance depended on the size of the recorded currents and the level of series resistance compensation that can safely be used. Following the achievement of whole cell configuration and sufficient for cell dialysis with pipette solution (>5 min), a standard voltage protocol was applied to the cell to evoke membrane currents. The voltage protocol is as follows. The membrane was depolarized from a holding potential of −80 mV to +20 mV for 1000 ms. This was followed by a descending voltage ramp (rate 0.5 mV msec⁻¹) back to the holding potential. The voltage protocol was applied to a cell continuously throughout the experiment every 4 seconds (0.25 Hz). The amplitude of the peak current elicited around −40 mV during the ramp was measured. Once stable evoked current responses were obtained in the external solution, vehicle (0.5% DMSO in the standard external solution) was applied for 10-20 min by a peristalic pump. Provided there were minimal changes in the amplitude of the evoked current response in the vehicle control condition, the test compound of either 0.3, 1, 3, 10 μM was applied for a 10 min period. The 10 min period included the time which supplying solution was passing through the tube from solution reservoir to the recording chamber via the pump. Exposing time of cells to the compound solution was more than 5 min after the drug concentration in the chamber well reached the attempting concentration. There reversibility. Finally, the cells was exposed to high dose of dofetilide (5 μM), a specific IKr blocker, to evaluate the insensitive endogenous current.

All experiments were performed at room temperature (23±1° C.). Evoked membrane currents were recorded on-line on a computer, filtered at 500-1 KHz (Bessel-3 dB) and sampled at 1-2 KHz using the patch clamp amplifier and a specific data analyzing software. Peak current amplitude, which occurred at around −40 mV, was measured off line on the computer.

The arithmetic mean of the ten values of amplitude was calculated under control conditions and in the presence of drug. Percent decrease of I_{N} in each experiment was obtained by the normalized current value using the following formula: I_{N}=(1−I_{D}/I_{C})×100, where I_{D} is the mean current value in the presence of drug and I_{C} is the mean current value under control conditions. Separate experiments were performed for each drug concentration or time-matched control, and arithmetic mean in each experiment is defined as the result of the study.

Mice PSL Method

Surgery of partial sciatic nerve ligation (PSL) was made according to Seltzer et al. (Pain 43, 1990, 205-218). Von Fray hair test was applied slowly to the plantar surface of the hind operated paw until the hairs bent. Each hair was tested 10 times in ascending order of force to different loci of the paw with one to two second intervals between each application. Once a withdrawal response was established, the paw was re-tested with the same hair. The lowest amount of force required to elicit a response was recorded as the paw-withdrawal threshold, measured in grams.

Chronic Contriction Injury Model (CCI Model):

Male Sprague-Dawley rats (270-300 g; B. W., Charles River, Tsukuba, Japan) were used.

The chronic constriction injury (CCI) operation was performed according to the method described by Bennett and Xie ¹⁾. Briefly, animals were anesthetized with sodium pentobarbital (64.8 mg/kg, i.p.) and the left common sciatic nerve was exposed at the level of the middle of the thigh by blunt dissection through biceps femoris. Proximal to the sciatic's trifurcation was freed of adhering tissue and 4 ligatures (4-0 silk) were tided loosely around it with about 1 mm space. Sham operation was performed as same as CCI surgery except for sciatic nerve ligation. Two weeks after surgery, mechanical allodynia was evaluated by application of von Frey hairs (VFHs) to the plantar surface of the hind paw. The lowest amount of force of VFH required to elicit a response was recorded as paw withdrawal threshold (PWT). VFH test was performed at 0.5, 1 and 2 hr post-dosing. Experimental data were analyzed using Kruskal-Wallis test followed by Dunn's test for multiple comparisons or Mann-Whitney U-test for paired comparison.

¹⁾ Bennett, G. J. and Xie, Y. K. Pain, 33:87-107, 1988

Serum Protein Binding

Serum protein binding of NR2B topic compounds (1 uM) in humans and ddY mice were measured in method of equilibrium dialysis using 96-well plate type equipment. Spectra-Por® regenerated cellulose membranes (molecular weight cut-off 12,000-14,000, 12 mm×120 mm) was soaked for over night in distilled water, then for 20 minutes in 30% ethanol, and finally for 15 minutes in dialysis buffer (0.10 M PBS: phosphate buffered saline, pH 7.4). Fresh humans and ddY mice serum (20 ml each) was prepared. The dialysis was assembled with being careful not to puncture or tear the membranes and added 150 ul of serum to one side of each well and 150 ul of dialysis buffer to the other side of each well. After 4 hours incubation at 37° C. for 60 r.p.m, remove the serum and buffer samples and an aliquot of collected serum and buffer samples were mixed for buffer and serum at following rates:

1) 40 ul serum samples were mixed with 120 ul buffer

2) 120 ul buffer samples were mixed with 40 ul serum

Then, mixed samples were extracted with 6001 μl acetonitrile containing CP-96344 at 25 ng/ml (as HPLC-MS-MS internal standard) and measured in LC/MS/MS analysis.

Calculations

The fraction of substrate unbound, fᵤ=1−{([plasma]_{eq}−[buffer]_{eq})/([plasma]_{eq})} where [plasma]_{eq} and [buffer]_{eq} are the concentrations of substrate in plasma and buffer, respectively.

Aqueous Solubility

Aqueous solubility in the mediums (a)-(c) was determined by method (1) or (2). (1) Vials containing approx. 1 mg of compound and 1 mL of each medium were agitated for 24 hours at room temperature. Insoluble materials were removed by centrifugation at 10,000 rpm for 10 minutes twice. The supernatants were assayed by HPLC. (2) Whatman Mini-UniPrep chambers (Clifton, N.J., USA) containing more than 0.5 mg of compound and 0.5 mL of each medium were shaken overnight (over 8 hours) at room temperature. All samples were filtered through a 0.45 μm PVDF membrane into a Whatman Mini-UniPrep plunger before analysis. The filtrates were assayed by HPLC.

<Mediums>

(a) Simulated gastric fluid with no enzyme (SGN) at pH 1.2: Dissolve 2.0 g of NaCl in 7.0 mL of ION HCl and sufficient water to make 1000 mL.

(b) Phosphate buffered saline (PBS) at pH 6.5: Dissolve 6.35 g of KH₂PO₄, 2.84 g of Na₂HPO₄ and 5.50 g of NaCl in sufficient water to make 1000 mL, adjusting the pH of this solution to 6.5.

(c) Water for injection (WFI).

Human V1a Binding Assay

Cell paste of CHO cells expressing human V1a receptor was suspended in 3-fold volume of ice-cold wash buffer (50 mM Tris-HCl, 5 mM MgCl₂, protease inhibitors, adjusted pH 7.4). The cells were homogenized and centrifuged at 25,000 g for 30 minutes at 4° C. The pellet was re-suspended by homogenization in freezing buffer (50 mM Tris-HCl, 5 mM MgCl₂, 20% glycerol, adjusted pH 7.4). The membrane homogenate was stored at −80° C. until use. All the manipulation was done on ice, and stock solution and equipment were kept on ice at all time.

For the saturation assay, receptor saturation was determined by incubating 8-Arg[phenylalanyl-3,4,5-³H]-vasopressin (³H-AVP) and 20 μg protein of cell membrane for 60 minutes at 25° C. in a final 250 μl of incubation buffer (50 mM Tris-HCl, 5 mM MgCl₂, 0.05% BSA, adjusted pH 7.4). Total and non-specific bindings (in the presence of 1 μM of d(CH₂)₅Tyr(Me)AVP [β-mercapto-β,β-cyclopentamethylene propionyl,O-Me-Tyr²,Arg⁸]-vasopressin (βMCPVP)) were determined in a range of ³H-AVP concentrations (0.05 nM to 100 nM).

For the competition assay, test compounds were incubated with 0.5 nM ³H-AVP and 20 μg protein of cell membrane for 60 minutes at 25° C. in a final 250 μl of incubation buffer (50 mM Tris-HCl, 5 mM MgCl₂, 0.05% BSA, adjusted pH 7.4). Nonspecific binding was determined by 1 μM of βMCPVP. The saturation derived K_{D} gained in saturation assay was used for all Ki calculations.

All incubations were terminated by filtration through Packard GF/C Unfilter plates pre-soaked in 0.5% polyethyleneimine followed by three washes with ice-cold filtration buffer (50 mM Tris-HCl, 5 mM MgCl₂, adjusted pH 7.4). The plates were then placed back into the incubator at 50° C. to dry. The bottom of the Unifilter plates were sealed using Packard plate seals and 50 μl of Microscint 0 was added to each well. The plates were then sealed with Packard Topseal A, and receptor-bound radioactivity was counted by Packard Topcount NXT.

For oral administration, tablets containing various excipients such as microcrystalline cellulose, sodium citrate, calcium carbonate, dipotassium phosphate and glycine may be employed along with various disintegrants such as starch and preferably corn, potato or tapioca starch, alginic acid and certain complex silicates, together with granulation binders like polyvinylpyrrolidone, sucrose, gelatin and acacia. Additionally, lubricating agents such as magnesium stearate, sodium lauryl sulfate and talc are often very useful for tabletting purposes. Solid compositions of a similar type may also be employed as fillers in gelatin capsules; preferred materials in this connection also include lactose or milk sugar as well as high molecular weight polyethylene glycols. When aqueous suspensions and/or elixirs are desired for oral administration, the active ingredient may be combined with various sweetening or flavoring agents, coloring matter or dyes, and, if so desired, emulsifying and/or suspending agents as well, together with such diluents as water, ethanol, propylene glycol, glycerin and various like combinations thereof.

For parenteral administration, solutions of a compound of the present invention in either sesame or peanut oil or in aqueous propylene glycol may be employed. The aqueous solutions should be suitably buffered (preferably pH>8) if necessary and the liquid diluent first rendered isotonic. These aqueous solutions are suitable for intravenous injection purposes. The oily solutions are suitable for intra-articular, intra-muscular and subcutaneous injection purposes. The preparation of all these solutions under sterile conditions is readily accomplished by standard pharmaceutical techniques well known to those skilled in the art. Additionally, it is also possible to administer the compounds of the present invention topically when treating inflammatory conditions of the skin and this may preferably be done by way of creams, jellies, gels, pastes, ointments and the like, in accordance with standard pharmaceutical practice.

### EXAMPLES

The invention is illustrated in the following non-limiting examples in which, unless stated otherwise: all operations were carried out at room or ambient temperature, that is, in the range of 18-25° C.; evaporation of solvent was carried out using a rotary evaporator under reduced pressure with a bath temperature of up to 60° C.; reactions were monitored by thin layer chromatography (tic) and reaction times are given for illustration only; melting points (m.p.) given are uncorrected (polymorphism may result in different melting points); the structure and purity of all isolated compounds were assured by at least one of the following techniques: tic (Merck silica gel 60 F₂₅₄ precoated TLC plates or Merck NH₂ F₂₅₄ₛ, precoated HPTLC plates), mass spectrometry, nuclear magnetic resonance (NMR), infrared red absorption spectra (IR) or microanalysis. Yields are given for illustrative purposes only. Flash column chromatography was carried out using Merck silica gel 60 (230-400 mesh ASTM) or Fuji Silysia Chromatorex® DU3050 (Amino Type, 30˜50 μm). Low-resolution mass spectral data (EI) were obtained on a Automass 120 (JEOL) mass spectrometer. Low-resolution mass spectral data (ESI) were obtained on a Quattro II (Micromass) mass spectrometer. NMR data were determined at 270 MHz (JEOL JNM-LA 270 spectrometer) or 300 MHz (JEOL JNM-LA300) using deuterated chloroform (99.8% D) or dimethylsulfoxide (99.9% D) as solvent unless indicated otherwise, relative to tetramethylsilane (TMS) as internal standard in parts per million (ppm); conventional abbreviations used are: s=singlet, d=doublet, t=triplet, q=quartet, m=multiplet, br. =broad, etc. IR spectra were measured by a Shimazu infrared spectrometer (IR-470). Optical rotations were measured using a JASCO DIP-370 Digital Polarimeter (Japan Spectroscopic CO, Ltd.).

Chemical symbols have their usual meanings; b.p. (boiling point), m.p. (melting point), I (liter(s)), ml (milliliter(s)), g (gram(s)), mg (milligram(s)), mol (moles), mmol (millimoles), eq. (equivalent(s)).

### Example 1

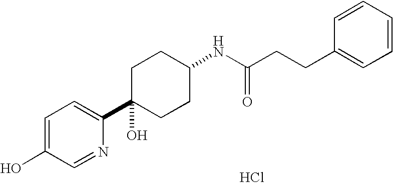

N-[cis-4-Hydroxy-4-(5-hydroxypyridin-2-yl)cyclohexyl]-3-phenylpropanamide Hydrochloride

1-A: N-(trans-4-Hydroxycyclohexyl)-3-phenylpropanamide

To a solution of trans-4-aminocyclohexanol (8.5 g, 74 mmol) in dichloromethane (200 ml) was added a solution of 3-phenylpropanoic acid (12 g, 81 mmol) in dichloromethane (96 ml). To this mixture were added WSC (16 g, 81 mmol) and HOBt (1.0 g, 7.4 mmol) at room temperature and the mixture was stirred overnight. The volatile materials were removed using a rotary evaporator under reduced pressure to give a residue, which was dissolved in chloroform (700 ml).

The solution was washed with sat. NaHCO₃ aq., dried over MgSO₄, and evaporated in vacuum. The residue was washed with diisopropyl ether (400 ml) to afford the titled compound as a white powder. (18 g, 97%)

¹H NMR (DMSO-d₆) δ: 7.62 (d, J=7.5 Hz, 1H), 7.29-7.12 (m, 5H), 4.51 (d, J=4.4 Hz, 1H), 3.52-3.27 (m, 2H), 2.78 (t, J=7.6 Hz, 2H), 2.31 (t, J=7.6 Hz, 2H), 1.82-1.63 (m, 4H), 1.25-1.03 (m, 4H) ppm.

1-B: N-(4-Oxocyclohexyl)-3-phenylpropanamide

To a solution of DMSO (2.5 g, 32 mmol) in dichloromethane (110 ml) was added oxalyl chloride (2.1 g, 16 mmol) dropwise at −60° C. After stirring 40 min, N-(trans-4-hydroxycyclohexyl)-3-phenylpropanamide (4.0 g, 16 mmol) was slowly added to the mixture. The mixture was warmed to −40° C. and stirred at −40° C. for 30 min. Triethylamine (5.4 g, 54 mmol) was added to the mixture at −40° C. and after 5 min, the mixture was warmed to room temperature. Water (100 ml) was added to the mixture and the organic layer was separated. The aqueous layer was extracted with ethyl acetate (30 ml×2). The combined extracts were washed with brine, dried over MgSO₄, and evaporated in vacuum. The residue was washed with diisopropyl ether to afford the titled compound as a white powder. (3.7 g, 94%)

¹H NMR (DMSO-d₆) δ: 7.86 (d, J=7.7 Hz, 1H), 7.38-7.11 (m, 5H), 4.11-3.96 (m, 1H), 2.82 (t, J=7.7 Hz, 2H), 2.50-2.14 (m, 4H), 2.38 (t, J=7.7 Hz, 2H), 2.07-1.87 (m, 2H), 1.70-1.49 (m, 2H) ppm.

1-C: N-[cis-4-Hydroxy-4-(5-hydroxypyridin-2-yl)cyclohexyl]-3-phenylpropanamide

To a solution of 6-bromopyridin-3-ol (11 g, 65 mmol) in THF (300 ml) was added dropwise a 1.6 M solution of n-butyllithium in hexane (42 ml, 65 mmol) at −78° C. and the mixture was stirred for 30 min. A 0.97M solution of sec-butyllitium in cyclohexane (100 ml, 97 mmol) was added dropwise and the mixture was stirred at −78° C. for 1 hour. To the mixture was added dropwise a solution of N-(4-oxocyclohexyl)-3-phenylpropanamide (5.3 g, 22 mmol) in THF (44 ml) at −78° C. and the mixture was stirred at −78° C. for 2 hours. Sat. NaH₂PO₄ aq. (150 ml) was slowly added to the mixture and the mixture was warmed to room temperature. The organic layer was separated and the aqueous layer was extracted with ethyl acetate (100 ml×3). The combined extracts were washed with brine, dried over MgSO₄, and evaporated in vacuum. The residue was purified by column chromatography on silica gel (dichloromethane:methanol=20:1 as eluent) to afford the titled compound as a white powder. (2.1 g, 29%)

¹H NMR (DMSO-d₆) δ: 9.68 (brs, 1H), 8.03 (d, J=2.6 Hz, 1H), 7.73 (d, J=7.7 Hz, 1H), 7.45 (d, J=8.6 Hz, 1H), 7.31-7.09 (m, 6H), 4.88 (s, 1H), 3.64-3.48 (m, 1H), 2.81 (t, J=7.6 Hz, 2H), 2.34 (t, J=7.6 Hz, 2H), 1.98-1.80 (m, 2H), 1.70-1.47 (m, 6H) ppm.

IR (KBr)vₘₐₓ: 3277, 2939, 2870, 1647, 1551, 1261 cm⁻¹.

MS (ESI): 339.21 (M−H)⁻

Anal. Calcd. for C20H24N2O3: C, 70.56; H, 7.11; N, 8.23. Found: C, 70.19; H, 7.11; N; 7.99.

1-D: N-[cis-4-Hydroxy-4-(5-hydroxypyridin-2-yl)cyclohexyl]-3-phenylpropanamide Hydrochloride

To a solution of N-[cis-4-hydroxy-4-(5-hydroxypyridin-2-yl)cyclohexyl]-3-phenylpropanamide (3.5 g, 10 mmol) in isopropanol (100 ml) was added 4 M solution of hydrogen chloride in ethyl acetate (2.9 ml, 11 mmol). The mixture was diluted with isopropanol (100 ml) and warmed to 50° C. to dissolve all the materials. The solution was filtered and the filtrate was concentrated (˜50 ml) to give a white powder. The resultant powder was filtered and dried in vacuum to afford the titled compound. (3.1 g, 79%)

¹H NMR (DMSO-d₆) δ: 11.77 (brs, 1H), 8.25 (s, 1H), 8.02-7.94 (m, 2H), 7.83 (d, J=7.7 Hz, 1H), 7.31-7.13 (m, 5H), 4.88 (s, 1H), 3.82-3.62 (m, 1H), 2.81 (t, J=7.7 Hz, 2H), 2.36 (t, J=7.7 Hz, 2H), 2.07-1.90 (m, 2H), 1.78-1.54 (m, 6H) ppm.

IR (KBr)vₘₐₓ: 3238, 2945, 2864, 1657, 1533, 1325, 995 cm⁻¹.

MS (ESI): 339.15 (M−H)⁻

### Example 2

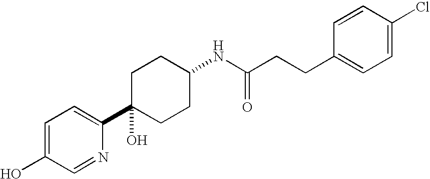

3-(4-Chlorophenyl)-N-[cis-4-hydroxy-4-(5-hydroxypyridin-2-yl)cyclohexyl]propanamide

2-A: 3-(4-Chlorophenyl)-N-(trans-4-hydroxycylohexyl)propanamide

The title compound was prepared from 3-(4-chlorophenyl)propanoic acid by the same manner as example 1-A.

¹H NMR (DMSO-d₆) δ: 7.63 (d, J=7.7 Hz, 1H), 7.31 (d, J=8.4 Hz, 2H), 7.20 (d, J=8.4 Hz, 2H), 4.51 (d, J=4.4 Hz, 1H), 3.50-3.26 (m, 2H), 2.77 (t, J=7.6 Hz, 2H), 2.30 (t, J=7.6 Hz, 2H), 1.85-1.58 (m, 4H), 1.26-1.01 (m, 4H) ppm.

2-B: 3-(4-Chlorophenyl)-N-(4-oxocyclohexyl)propanamide

The title compound was prepared from 3-(4-chlorophenyl)-N-(trans-4-hydroxycyclohexyl)propanamide by the same manner as example 1-B.

¹H NMR (DMSO-d₆) δ: 7.86 (d, J=7.1 Hz, 1H), 7.42-7.18 (m, 4H), 4.10-3.92 (m, 1H), 2.81 (t, J=7.7 Hz, 2H), 2.50-2.16 (m, 4H), 2.37 (t, J=7.7 Hz, 2H), 2.05-1.86 (m, 2H), 1.70-1.50 (m, 2H) ppm.

2-C: 3-(4-Chlorophenyl)-N-[cis-4-hydroxy-4-(5-hydroxypyridin-2 yl)cyclohexyl]propanamide

The title compound was prepared from 3-(4-chlorophenyl)-N-(4-oxocyclohexyl)propanamide by the same manner as example 1-C.

¹H NMR (DMSO-d₆) δ: 9.68 (s, 1H), 8.02 (d, J=2.9 Hz, 1H), 7.74 (d, J=7.7 Hz, 1H), 7.45 (d, J=8.6 Hz, 1H), 7.35-7.29 (m, 2H), 7.26-7.20 (m, 2H), 7.15-7.10 (m, 1H), 4.88 (s, 1H), 3.64-3.48 (m, 1H), 2.80 (t, J=7.6 Hz, 2H), 2.33 (t, J=7.6 Hz, 2H), 1.98-1.80 (m, 2H), 1.69-1.49 (m, 6H) ppm.

IR (KBr)vₘₐₓ: 3250, 2939, 2862, 1645, 1553, 1493 cm⁻¹.

MS (ESI): 375.38 (M+H)⁺, 373.38 (M−H)⁻

### Example 3

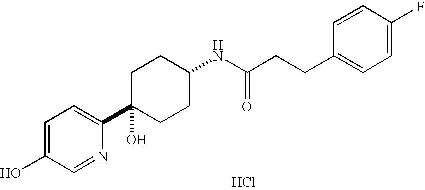

3-(4-Fluorophenyl)-N-[cis-4-hydroxy-4-(5-hydroxypyridin-2-yl)cyclohexvl]propanamide Hydrochloride

3-A: 6-(8-Hydroxy-1,4-dioxaspiro[4.5]dec-8-yl)pyridin-3-ol

To a solution of 6-bromopyridin-3-ol (8.0 g, 46 mmol) in THF (150 ml) was added dropwise a 1.4 M solution of n-butyllithium in hexane (33 ml, 46 mmol) at −78° C. and the mixture was stirred for 20 min. A 0.90 M solution of sec-butyllitium in cyclohexane (77 ml, 69 mmol) was added dropwise and the mixture was stirred at −78° C. for 1 hour. To the mixture was added a solution of 1,4-dioxaspiro[4.5]decan-8-one (11 g, 69 mmol) in THF (70 ml) dropwise at −78° C. and the mixture was stirred at −78° C. for 1 hour. Sat. NaH₂PO₄ aq. (100 ml) was slowly added to the mixture and the mixture was warmed to room temperature. The organic layer was separated and the aqueous layer was extracted with ethyl acetate (100 ml×3). The combined extracts were washed with brine, dried over MgSO₄, and evaporated in vacuum. The residue was purified by column chromatography on silica gel (hexane:ethyl acetate=1:2 as eluent) to afford the titled compound as a white powder. (9.1 g, 79%)

¹H NMR (DMSO-d₆) δ: 9.67 (s, 1H), 8.02 (d, J=2.9 Hz, 1H), 7.45 (d, J=8.5 Hz, 1H), 7.12 (dd, J=8.5, 2.9 Hz, 1H), 4.93 (s, 1H), 3.87 (s, 4H), 2.22-2.04 (m, 2H), 1.97-1.81 (m, 2H), 1.61-1.44 (m, 4H) ppm.

3-B: 8-[5-(Benzyloxy)pyridin-2-yl]-1,4-dioxaspiro[4.5]decan-8-ol

To a solution of 6-(8-hydroxy-1,4-dioxaspiro[4.5]dec-8-yl)pyridin-3-ol (0.95 g, 3.8 mmol) in THF (4.0 ml) was added NaH (60% in oil, 0.38 g, 9.5 mmol) portionwise at 0° C. The mixture was stirred at 0° C. for 30 min. To the mixture was added a solution of benzylbromide (0.71 g, 4.2 mmol) in DMSO (4.0 ml) slowly at 0° C. The mixture was stirred at 0° C. for 30 min and at room temperature for additional 2 hours. H₂O (30 ml) was slowly added to the mixture and the organic layer was separated. The aqueous layer was extracted with ethyl acetate (15 ml×3). The combined extracts were washed with brine, dried over MgSO₄, and evaporated in vacuum to afford the titled compound as a white powder. (1.3 g, 99%)

¹H NMR (DMSO-d₆) δ: 8.13 (d, J=3.1 Hz, 1H), 7.50-7.17 (m, 7H), 5.05 (s, 2H), 4.90 (s, 1H), 3.76 (s, 4H), 2.12-1.95 (m, 2H), 1.86-1.70 (m, 2H), 1.50-1.35(m, 4H) ppm.

3-C: 4-[5-(Benzyloxy)pyridin-2-Y11-4-hydroxycyclohexanone

To a solution of 8-[5-(benzyloxy)pyridin-2-yl]-1,4-dioxaspiro[4.5]decan-8-ol (1.3 g, 1.4 mmol) in THF (40 ml) was added 2 M HCl aq. (20 ml). The mixture was stirred at 50° C. for 2 hours. THF was removed in vacuum and the residue was made basic with 2 M NaOH aq. (25 ml). The mixture was extracted with ethyl acetate (40 ml×3). The combined extracts were washed with brine, dried over MgSO₄, and evaporated in vacuum. The residue was purified by column chromatography on silica gel (hexane:ethyl acetate=3:1 as eluent) to afford the titled compound as a white powder. (0.95 g, 84%)

¹H NMR (DMSO-d₆) δ: 8.27 (d, J=2.9 Hz, 1H), 7.66 (d, J=8.8 Hz, 1H), 7.53-7.31 (m, 6H), 5.52 (s, 1H), 5.18 (s, 2H), 2.80-2.64 (m, 2H), 2.44-2.24 (m, 2H), 2.20-2.10 (m, 2H), 1.96-1.85 (m, 2H) ppm.

3-D: N-{cis-4-[5-(Benzyloxy)pyridin-2-yl]-4-hydroxycyclohexyl}-3-(4-fluorophenyl)propanamide

To a solution of 4-[5-(benzyloxy)pyridin-2-yl]-4-hydroxycyclohexanone (0.35 g, 1.0 mmol) in methanol (7.0 ml) was added ammonium acetate (0.79 g, 10 mmol) at room temperature. The mixture was stirred for 2 hours. To the mixture was added NaBH₃CN (0.16 g, 2.6 mmol) at 0° C. and the mixture was stirred at 0° C. for 1 hour. Methanol was removed in vacuum to give a residue, which was diluted with 2 M NaOH aq. (4.0 ml). The mixture was extracted with ethyl acetate (10 ml×3). The combined extracts were washed with brine, dried over MgSO₄, and evaporated in vacuum to afford a white powder. To a solution of the powder in dichloromethane (5.1 ml) were added 3-(4-fluorophenyl)propanoic acid (0.21 g, 1.2 mmol), WSC (0.22 g, 1.1 mmol) and HOBt (0.014 g, 0.10 mmol). The mixture was stirred at room temperature overnight. Sat. NaHCO₃ aq. (10 ml) was added to the mixture and the mixture was extracted with ethyl acetate (10 ml×3). The combined extracts were washed with brine, dried over MgSO₄, and evaporated in vacuum. The residue was purified by column chromatography on silica gel (dichloromethane: methanol=50: 1 as eluent) to afford the titled compound as a white powder. (0.14 g, 30%)

¹H NMR (CDCl₃) δ: 8.25 (d, J=2.6 Hz, 1H), 7.49-7.12 (m, 9H), 7.04-6.91 (m, 2H), 5.30-5.21 (m, 1H), 5.11 (s, 2H), 4.98 (br, 1H), 3.98-3.80 (m, 1H), 2.95 (t, J=7.5 Hz, 2H), 2.43 (t, J=7.5 Hz, 2H), 1.90-1.52 (m, 8H) ppm.

3-E: 3-(4-Fluorophenyl)-N-[cis-4-hydroxy-4-(5-hydroxypyridin-2-yl)cyclohexyl]propanamide

To a solution of N-{cis-4-[5-(benzyloxy)pyridin-2-yl]-4-hydroxycyclohexyl}-3-(4-fluorophenyl)propanamide (0.14 g, 0.31 mmol) in methanol (14 ml) was added Pd-C (10%, 0.033 g, 0.0031 mgatm). The reactor was charged with H₂ (1 atm) and the mixture was stirred at 40° C. for 6 hours. The mixture was filtered though a pad of celite and the filtrate was evaporated in vacuum. The residue was purified by column chromatography on silica gel (dichloromethane: methanol=20:1 as eluent) to afford the titled compound as a white powder. (0.10 g, 89%)

¹H NMR (DMSO-d₆) δ: 9.68 (brs, 1H), 8.02 (d, J=2.7 Hz, 1H), 7.72 (d, J=8.1 Hz, 1H), 7.44 (d, J=8.6 Hz, 1H), 7.28-7.18 (m, 2H), 7.15-7.03 (m, 3H), 4.88 (s, 1H), 3.64-3.48 (m, 1H), 2.79 (t, J=7.7 Hz, 2H), 2.33 (t, J=7.7 Hz, 2H), 1.96-1.80 (m, 2H), 1.69-1.47 (m, 6H) ppm.

3-F: 3-(4-Fluorophenyl)-N-[cis-4-hydroxy-4-(5-hydroxypyridin-2-yl)cyclohexyl]propanamide Hydrochloride

The title compound was prepared from 3-(4-fluorophenyl)-N-[cis-4-hydroxy-4-(5-hydroxypyridin-2-yl)cyclohexyl]propanamide by the same manner as example 1-D.

¹H NMR (DMSO-d₆) δ: 11.61 (brs, 1H), 8.22-8.17 (m, 1H), 8.01-7.89 (m, 2H), 7.84-7.76 (m, 1H), 7.29-7.18 (m, 2H), 7.14-7.04 (m, 2H), 3.77-3.63 (m, 1H), 2.80 (t, J=7.6 Hz, 2H), 2.34 (t, J=7.6 Hz, 2H), 2.05-1.87 (m, 2H), 1.76-1.56 (m, 6H) ppm. (—OH was not observed)

IR (KBr)vₘₐₓ: 3275, 3082, 2947, 1647, 1541, 1508 cm⁻¹.

MS (ESI): 359.22 (M+H)⁺

### Example 4

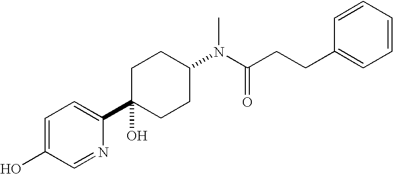

N-[cis-4-Hydroxy-4-(5-hydroxypyridin-2-yl)cyclohexyl]-N-methyl-3-phenylpropanamide

4-A: cis-1-[5-(benzyloxy)pyridin-2-yl]-4-(methylamino)cyclohexanol

A mixture of 4-[5-(benzyloxy)pyridin-2-yl]-4-hydroxycyclohexanone (5.0 g, 16.8 mmol) and methylamine (40% in methanol, 3.9 g, 50 mmol) in methanol (100 ml) was stirred at room temperature overnight. After cooling to −30° C., NaBH₄ (0.64 g, 17 mmol) was added to the mixture. The mixture was stirred at room temperature (to 0° C.) for 3 hours. (ca.50 g) was added and the solvent was removed in vacuum. The residue was purified by a short column on silica gel (NH-gel, dichloromethane: methanol=20:1 as eluent). After concentration, the precipitates were triturated with ether. The solid was filtered to afford the titled compound as a white powder. (4.3 g, 81%)

¹H NMR (CDCl₃) δ: 8.23 (s, 1H), 7.49-7.26 (m, 7H), 5.12 (s, 2H), 4.61 (brs, 1H), 2.55-2.41 (m, 4H), 2.00-1.56 (m, 8H) ppm. (—OH was not observed)

4-B: N-{cis-4-[5-(Benzyloxy)pyridin-2-yl]-4-hydroxycyclohexyl}-N-methyl-3-phenylpropanamide

To a solution of cis-1-[5-(benzyloxy)pyridin-2-yl]-4-(methylamino)cyclohexanol (0.44 g, 1.4 mmol) in dichloromethane (7.0 ml) were added 3-phenylpropanoyl chloride (0.45 g, 2.7 mmol) and triethylamine (0.41 g, 4.0 mmol) at 0° C. The mixture was stirred at 0° C. for 30 min and at room temperature for an additional 1 hour. Sat. NaHCO₃ aq. was added to the mixture and the organic layer was separated. The aqueous layer was extracted with dichloromethane (15 ml×2). The combined extracts were washed with brine, dried over MgSO₄, and evaporated in vacuum. The residue was purified by column chromatography on silica gel (hexane:ethyl acetate=1:1 to 1:4 as eluent) to afford the titled compound as a white powder. (0.46 g, 76%)

¹H NMR (DMSO-d₆) δ: 8.29-8.21 (m, 1H), 7.61-7.54 (m, 1H), 7.49-7.12 (m, 11H), 5.19-5.13 (m, 2H), 5.02 (s, 1H), 4.48-4.31 and 3.79-3.64 (m, 1H), 2.86-2.54 (m, 4H), 2.80 and 2.73 (s, 3H), 2.09-1.85 (m, 4H), 1.65-1.53 (m, 2H), 1.42-1.27 (m, 2H) ppm.

4-C: N-[cis-4-Hydroxy-4-(5-hydroxypyridin-2-yl)cyclohexyl]-N-methyl-3-phenylpropanamide

The title compound was prepared from N-{cis-4-[5-(benzyloxy)pyridin-2-yl]-4-hydroxycyclohexyl}-N-methyl-3-phenylpropanamide by the same manner as example 3-E.

¹H NMR (DMSO-d₆) δ: 9.69 (s, 1H), 8.07-7.99 (m, 1H), 7.50-7.43 (m, 1H), 7.32-7.10 (m, 6H), 4.95 (s, 1H), 4.46-4.32 and 3.79-3.64 (m, 1H), 2.86-2.54 (m, 7H), 2.08-1.80 (m, 4H), 1.66-1.51 (m, 2H), 1.45-1.26 (m, 2H) ppm.

IR (KBr)vₘₐₓ: 3468, 3171, 2920, 2862, 1605, 1583, 1265 cm⁻¹.

MS (ESI): 355.01 (M+H)⁺, 352.94 (M−H)⁻

### Example 5

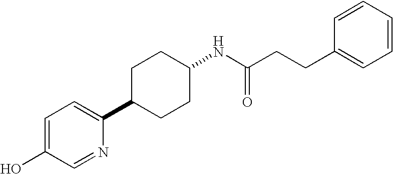

N-[trans-4-(5-Hydroxypyridin-2-yl)cyclohexyl]-3-phenylpropanamide Hydrochloride

5-A: 5-(Benzyloxy)-2-(1,4-dioxaspiro[4.5]dec-7-en-8-yl)pyridine

To a solution of 6-(8-hydroxy-1,4-dioxaspiro[4.5]dec-8-yl)pyridin-3-ol (3.5 g, 10 mmol, 3-B) in benzene (100 ml) was added (methoxycarbonylsulfamoyl)triethylammonium hydroxide, inner salt (3.7 g, 15 mmol). The mixture was stirred at 85° C. for 30 min. Sat. NaHCO₃ aq. (40 ml) was added to the mixture and the organic layer was separated. The aqueous layer was extracted with ethyl acetate (50 ml×3). The combined extracts were washed with brine, dried over MgSO₄, and evaporated in vacuum. The residue was purified by column chromatography on silica gel (hexane:ethyl acetate=4:1 as eluent) to afford the titled compound as a white powder. (2.1 g, 64%)

¹H NMR (CDCl₃) δ: 8.31 (d, J=2.8 Hz, 1H), 7.47-7.15 (m, 7H), 6.46-6.38 (m, 1H), 5.10 (s, 2H), 4.02 (s, 4H), 2.78-2.69 (m, 2H), 2.53-2.45 (m, 2H), 1.95-1.87 (m, 2H) ppm.

5-B: 4-[5-(Benzyloxy)pyridin-2-yl]cyclohexanone

To a solution of 5-(benzyloxy)-2-(1,4-dioxaspiro[4.5]dec-7-en-8-yl)pyridine (2.1 g, 6.5 mmol) in methanol (65 ml) was added Pd-C (10%, 0.69 g, 0.65 mgatm). The reactor was charged with H₂ (1 atm) and the mixture was stirred at room temperature for 3 hours. The mixture was filtered though a pad of celite and the filtrate was evaporated in vacuum. The residue was dissolved into THF (7.0 ml) and NaH (60% in oil, 0.39 g, 9.8 mmol) was added to the solution portionwise at 0° C. The mixture was stirred at 0° C. for 30 min. To the mixture was added a solution of benzylbromide (1.2 g, 7.2 mmol) in DMSO (7.0 ml) slowly at room temperature. The mixture was stirred at room temperature for 2 hours. H₂O (30 ml) was slowly added to the mixture and the organic layer was separated. The aqueous layer was extracted with ethyl acetate (20 ml×3). The combined extracts were washed with brine, dried over MgSO₄, and evaporated in vacuum. The residue was dissolved into THF (65 ml) and 2 M HCl aq. (32 ml) was added to the solution. The mixture was stirred at 50° C. for 2 hours. THF was removed in vacuum and the residue was made basic with 2 M NaOH aq. (40 ml). The mixture was extracted with dichloromethane (40 ml×3). The combined organic layers were washed with brine, dried over MgSO₄, and evaporated in vacuum. The residue was purified by column chromatography on silica gel (hexane:ethyl acetate=2:1 as eluent) to afford the titled compound as a white powder. (1.6 g, 89%)

¹H NMR (CDCl₃) δ: 8.32 (d, J=2.8 Hz, 1H), 7.48-7.33 (m, 5H), 7.26-7.19 (m, 1H), 7.11 (d, J=8.7 Hz, 1H), 5.10 (s, 2H), 3.25-3.08 (m, 1H), 2.58-2.46 (m, 4H), 2.34-2.21 (m, 2H), 2.12-1.94 (m, 2H) ppm.

5-C: N-{trans-4-[5-(Benzyloxy)pyridin-2-yl]cyclohexyl}-3-phenylpropanamide

To a solution of 4-[5-(benzyloxy)pyridin-2-yl]cyclohexanone (0.50 g, 1.8 mmol) in methanol (10 ml) was added ammonium acetate (1.4 g, 18 mmol) at room temperature. The mixture was stirred for 2 hours. To the mixture was added NaBH₃CN (0.16 g, 2.6 mmol) at 0° C. and the mixture was stirred at 0° C. for 40 min and at room temperature overnight. Methanol was removed in vacuum to give a residue, which was diluted with 2 M NaOH aq. (10 ml). The mixture was extracted with dichloromethane (30 ml×3). The combined organic layers were washed with brine, dried over MgSO₄, and evaporated in vacuum to afford a white powder. The powder was dissolved in dichloromethane (5.7 ml) and to this solution were added 3-phenylpropanoic acid (0.20 g, 1.4 mmol), WSC (0.24 g, 1.2 mmol) and HOBt (0.015 g, 0.11 mmol). The mixture was stirred at room temperature overnight. Sat. NaHCO₃ aq. was added to the mixture and the mixture was extracted with dichloromethane (10 ml×3). The combined extracts were washed with brine, dried over MgSO₄, and evaporated in vacuum. The residue was purified by column chromatography on silica gel (dichloromethane: methanol=50:1 as eluent) to afford the titled compound as a white powder. (0.25 g, 53%)

¹H NMR (CDCl₃) δ: 8.28 (d, J=2.9 Hz, 1H), 7.48-7.16 (m, 11H), 7.04 (d, J=8.6 Hz, 1H), 5.18-5.06 (m, 1H), 5.08 (s, 2H), 3.89-3.73 (m, 1H), 2.97 (t, J=7.6 Hz, 2H), 2.66-2.53 (m, 1H), 2.45 (t, J=7.6 Hz, 2H), 2.10-1.91 (m, 4H), 1.67-1.50 (m, 2H), 1.23-1.07 (m, 2H) ppm.

5-D: N-[trans-4-(5-Hydroxypyridin-2-yl)cyclohexyl]-3-phenylpropanamide

The title compound was prepared from N-{trans-4-[5-(benzyloxy)pyridin-2-yl]cyclohexyl}-3-phenylpropanamide by the same manner as example 3-E.

¹H NMR (DMSO-d₆) δ: 9.58 (s, 1H), 8.04-8.00 (m, 1H), 7.71 (d, J=7.7 Hz, 1H), 7.30-7.13 (m, 5H), 7.08-7.04 (m, 2H), 3.63-3.47 (m, 1H), 2.80 (t, J=7.7 Hz, 2H), 2.56-2.44 (m, 1H), 2.34 (t, J=7.7 Hz, 2H), 1.88-1.75 (m, 4H), 1.60-1.41 (m, 2H), 1.32-1.13 (m, 2H) ppm.

MS (ESI): 325.13 (M+H)⁺, 323.06 (M−H)⁻

5-E: N-[trans-4-(5-Hydroxypyridin-2-yl)cyclohexyl]-3-phenylpropanamide Hydrochloride

The title compound was prepared from N-[trans-4-(5-hydroxypyridin-2-yl)cyclohexyl]-3-phenylpropanamide by the same manner as example 1-D.

¹H NMR (DMSO-d₆) δ: 11.58 (brs, 1H), 8.27-8.21 (m, IH), 7.97-7.90 (m, 1H), 7.83-7.77 (m, 2H), 7.31-7.13 (m, 5H), 3.70-3.54 (m, 1H), 2.99-2.84 (m, 1H), 2.81 (t, J=7.6 Hz, 2H), 2.35 (t, J=7.6 Hz, 2H), 1.94-1.80 (m, 4H), 1.75-1.58 (m, 2H), 1.33-1.15 (m, 2H) ppm.

IR (KBr)vₘₐₓ: 3404, 2934, 2862, 1618, 1560, 1452, 1308 cm⁻¹.

MS (ESI): 325.18 (M+H)⁺, 323.14 (M−H)⁻

### Example 6

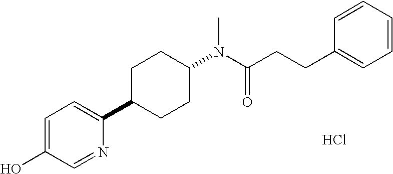

N-[trans-4-(5-Hydroxypyridin-2-yl)cyclohexyl]-N-methyl-3-phenylpropanamide Hydrochloride

6-A: trans-4-[5-(benzyloxy)pyridin-2-yl]-N-methylcyclohexnamine

To a suspension of 4-[5-(benzyloxy)pyridin-2-yl]-4-hydroxycyclohexanone (0.40 g, 1.4 mmol, 3-C) in ethanol (14 ml) was added a solution of methylamine (40% in methanol, 1.5 ml, 14 mmol) at 0° C. and the mixture was stirred at room temperature overnight. To the mixture was added NaBH₄ (0.11 g, 2.8 mmol) at 0° C. The mixture was stirred at 0° C. for 1 hour. 2 M HCl aq. (4.0 ml) was slowly added to the mixture at 0° C. and the mixture was warmed to room temperature. The mixture was made basic with 2 M NaOH aq. (5.0 ml). The mixture was extracted with ethyl acetate (15 ml×3). The combined organic layers were washed with brine, dried over MgSO₄, and evaporated in vacuum. The residue was purified by column chromatography on NH-gel (dichloromethane:methanol=100:1 as eluent) to afford the titled compound as a white powder. (0.34 g, 65%)

¹H NMR (CDCl₃) δ: 8.30 (d, J=2.4 Hz, 1H), 7.47-7.30 (m, 5H), 7.23-7.15 (m, 1H), 7.06 (d, J=8.3 Hz, 1H), 5.08 (s, 2H), 2.71-2.58 (m, 1H), 2.50-2.35 (m, 1H), 2.46 (s, 3H), 2.13-1.90 (m, 4H), 1.66-1.42 (m, 2H), 1.30-1.23 (m, 2H) ppm. (—NH was not observed)

6-B: N-{trans-4-[5-(benzyloxy)pyridin-2-yl]cyclohexyl}-N-methyl-3-phenylpropanamide

To a solution of trans-4-[5-(benzyloxy)pyridin-2-yl]-N-methylcyclohexnamine (0.097 g, 0.32 mmol) in dichloromethane (3.0 ml) were added 3-phenylpropanoic acid (0.058 g, 0.39 mmol), WSC (0.068 g, 0.35 mmol) and HOBt (0.0043 g, 0.032 mmol). The mixture was stirred at room temperature overnight. Sat. NaHCO₃ aq. was added to the mixture and the mixture was extracted with dichloromethane (5.0 ml×3). The combined extracts were washed with brine, dried over MgSO₄, and evaporated in vacuum. The residue was purified by column chromatography on silica gel (dichloromethane:methanol=50:1 as eluent) to afford the titled compound as a white powder. (0.12 g, 87%)

¹H NMR (CDCl₃) δ: 8.34-8.28 (m, 1H), 7.50-7.00 (m, 12H), 5.08 (s, 2H), 4.69-4.53 and 3.70-3.54 (m, 1H), 3.06-2.90 (m, 2H), 2.85 and 2.78 (s, 3H), 2.74-2.52 (m, 3H), 2.11-1.91 (m, 2H), 1.81-1.44 (m, 6H) ppm.

6-C: N-[trans-4-(5-Hydroxypyridin-2-yl)cyclohexyl]-N-methyl-3-phenylpropanamide

The title compound prepared was from N-{trans-4-[5-(benzyloxy)pyridin-2-yl]cyclohexyl}-N-methyl-3-phenylpropanamide by the same manner as example 3-E.

¹H NMR (DMSO-d₆) δ: 9.59 (s, 1H), 8.04-7.98 (m, 1H), 7.32-7.13 (m, 5H), 7.09-7.03 (m, 2H), 4.42-4.28 and 3.72-3.60 (m, 1H), 2.78 and 2.71 (s, 3H), 2.86-2.50 (m, 5H), 1.93-1.77 (m, 2H), 1.72-1.46 (m, 6H) ppm.

MS (ESI): 339.19 (M+H)⁺, 337.14 (M−H)⁻

6-D:

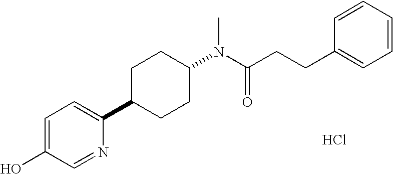

N-[trans-4-(5-Hydroxypyridin-2-yl)cyclohexyl]-N-methyl-3-phenylpropanamide Hydrochloride

The title compound was prepared from N-[trans-4-(5-hydroxypyridin-2-yl)cyclohexyl]-N-methyl-3-phenylpropanamide by the same manner as example 1-D.

¹H NMR (DMSO-d₆) δ: 11.52 (brs, 1H), 8.26-8.21 (m, 1H), 7.95-7.87 (m, 1H), 7.82-7.68 (m, 1H), 7.32-7.14 (m, 5H), 4.48-4.34 and 3.78-3.64 (m, 1H), 2.80 and 2.72 (s, 3H), 2.98-2.54 (m, 5H), 2.02-1.88 (m, 2H), 1.83-1.54 (m, 6H) ppm.

IR (KBr)vₘₐₓ: 3396, 2934, 2648, 2513, 1595, 1553, 1331 cm⁻¹.

MS (ESI): 339.24 (M+H⁺), 337.18 (M−H⁻)

### Example 7

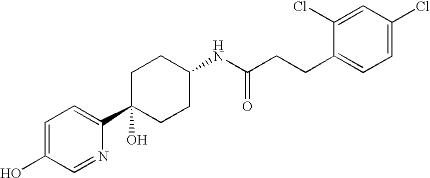

3-(2,4-dichlorophenyl)-N-[cis-4-hydroxy-4-(5-hydroxypyridin-2-yl)cyclohexyl]propanamide

7-A: cis-4-(benzylamino)-1-[5-(benzyloxy)pyridin-2-yl]cyclohexanol

A mixture of 4-[5-(benzyloxy)pyridin-2-yl]-4-hydroxycyclohexanone (5.0 g, 17 mmol) and benzylamine (5.7 ml, 51 mmol) in methanol (100 ml) was stirred at room temperature overnight. After cooling to −30° C., NaBH₄ (0.64 g, 17 mmol) was added to the mixture. The mixture was stirred at 0° C. for 3 hours. NH-gel (ca.50 g) was added and the solvent was removed in vacuum. The residue was purified by a short column on NH-gel, eluting with dichloromethane-methanol (20:1). After concentration, the precipitates were triturated with diisopropylether. The solid was filtered to afford the titled compound. (5.00 g, 77%)

¹H NMR (CDCl₃) δ: 8.28 (t, J=1.8 Hz, 1H), 7.49-7.21 (m, 12H), 5.12 (s, 2H), 4.58 (brs, 1H), 3.91 (s, 2H), 2.69-2.58 (m, 1H), 2.02-1.50 (m, 8H) ppm. (—OH was not observed)

7-B: 6-(cis-4-amino-1-hydroxycyclohexyl)pyridin-3-ol

To a solution of cis-4-(benzylamino)-1-[5-(benzyloxy)pyridin-2-yl]cyclohexanol (1.8 g, 0.31 mmol) in methanol (100 ml) was added Pd(OH)₂—C (20%, 0.9 g). The reactor was charged with H₂ (4 atm) and the mixture was stirred at room temperature for 7 hours. THF (150 ml) was added and the mixture was stirred for 30 min at reflux temperature. The mixture was filtered though a pad of celite and the filtrate was evaporated in vacuum to afford the titled compound. (1.2 g, quant)

¹H NMR (DMSO-d₆) δ: 8.02-7.96 (m, 1H), 7.45-7.37 (m, 1H), 7.13-7.03 (m, 1H), 4.80 (brs, 1H), 3.63-3.21 (m, 1H), 2.59-2.22 (m, 1H), 1.97-1.33 (m, 8H) ppm. (—OH and —NH₂ were not observed)

7-C: 3-(2,4-dichlorophenyl)-N-[cis-4-hydroxy-4-(5-hydroxypyridin-2-yl)cyclohexyl]propanamide

A mixture of 6-(cis-4-amino-1-hydroxycyclohexyl)pyridin-3-ol (0.48 g, 2.3 mmol), 3-(2,4-dichlorophenyl)propanoic acid (1.0 g, 4.6 mmol), WSC(0.89 g, 4.6 mmol) and HOBt (0.03 g, 0.23 mmol) in DMF (4.0 ml) was stirred at room temperature overnight. The mixture was quenched with H₂O and extracted with ethyl acetate (50 ml×2). The combined extracts were dried over MgSO₄ and concentrated in vacuum to afford the yellow solid. This solid was treated with 1M NaOH (10 ml) in methanol (60 ml) at room temperature for 30 min. After the mixture was evaporated in vacuum, the residue was neutralized with 2 M HCl and extracted with ethyl acetate (50 ml×2). The combined extracts were washed sat. NaHCO₃ aq., dried over MgSO₄ and concentrated in vacuum. The obtained solid was washed with dichloromethane to afford the titled compound. (0.46 g, 51%)

¹H NMR (DMSO-d₆) δ: 9.73 (brs, 1H), 8.02 (d, J=2.7 Hz, 1H), 7.82 (d, J=7.8 Hz, 1H), 7.63-7.53 (m, 1H), 7.45 (d, J=8.7 Hz, 1H), 7.40-7.30 (m, 2H), 7.19-7.08 (m, 1H), 4.92 (s, 1H), 3.65-3.25 (m, 1H), 2.98-2.84 (m, 2H), 2.43-2.28 (m, 2H), 1.97-1.79 (m, 2H), 1.73-1.45 (m, 6H) ppm. (OH and NH were not observed)

MS (ESI): 410.8 (M+H)⁺, 408.9 (M−H)⁻

IR (KBr)vₘₐₓ: 3630, 3290, 1645, 1553, 1474, 1431, 1261, 1138, 1099, 1055, 982, 839, 826, 766 cm⁻¹.

m.p. 198.1° C.

### Example 8

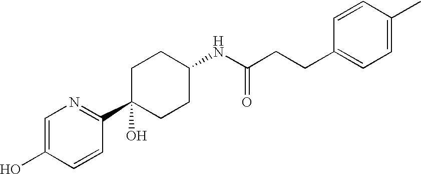

N-[cis-4-hydroxy-4-(5-hydroxypyridin-2-yl)cyclohexyl]-3-(4-methylphenyl)propanamide

The title compound was prepared from 6-(cis-4-amino-1-hydroxycyclohexyl)pyridin-3-ol and 3-(4-methylphenyl)propionic acid by the same manner as example 7-C.

¹H NMR (DMSO-d₆) δ: 9.69 (brs, 1H), 8.01 (d, J=2.8 Hz, 1H), 7.73 (d, J=7.7 Hz, 1H), 7.45-7.03 (m, 6H), 4.89 (s, 1H), 3.63-3.48 (m, 1H), 2.75 (t, J=7.2 Hz, 2H), 2.30 (t, J=7.2 Hz, 2H), 2.25 (s, 3H), 1.96-1.81 (m, 2H), 1.70-1.47 (m, 6H) ppm.

IR (KBr)vₘₐₓ: 3273, 1645, 1547, 1286, 837 cm⁻¹.

MS (ESI): 355.0 (M+H)⁺, 353.0 (M−H)⁻

### Example 9

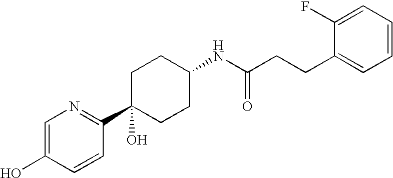

3-(2-fluorophenyl)-N-[cis-4-hydroxy-4-(5-hydroxypyridin-2-yl)cyclohexyl]propanamide

The title compound was prepared from 6-(cis-4-amino-1-hydroxycyclohexyl)pyridin-3-ol and 3-(2-fluorophenyl)propionic acid by the same manner as example 7-C.

¹H NMR (DMSO-d₆) δ: 9.70 (brs, 1H), 8.02 (d, J=2.4 Hz, 1H), 7.77 (d, J=8.1 Hz, 1H), 7.45 (d, J=8.6 Hz, 1H), 7.32-7.06 (m, 5H), 4.89 (s, 1H), 3.63-3.48 (m, 1H), 2.83 (t, J=8.1 Hz, 2H), 2.30 (t, J=8.1 Hz, 2H), 1.96-1.81 (m, 2H), 1.70-1.47 (m, 6H) ppm.

IR (KBr)vₘₐₓ: 3265, 1643, 1543, 1493, 1288, 833, 762 cm⁻¹.

MS (ESI): 359.0 (M+H)⁺, 357.0 (M−H)⁻

### Example 10

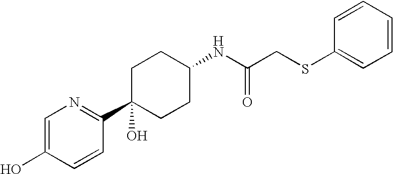

N-[cis-4-hydroxy-4-(5-hydroxypyridin-2-yl)cyclohexyl]-2-(phenylthio)acetamide

The title compound was prepared from 6-(cis-4-amino-1-hydroxycyclohexyl)pyridin-3-ol and 2-(phenylthio)acetic acid by the same manner as example 7-C.

¹H NMR (DMSO-d₆) δ: 9.72 (brs, 1H), 8.07-8.00 (m, 2H), 7.48-7.27 (m, 5H), 7.21-7.09 (m, 2H), 4.91 (s, 1H), 3.63-3.48 (m, 3H), 1.96-1.81 (m, 2H), 1.72-1.47 (m, 6H) ppm.

IR (KBr)vₘₐₓ: 3271, 1649, 1553, 1288, 1207, 741 cm⁻¹.

MS (ESI): 359.15 (M+H)⁺, 357.11 (M−H)⁻

### Example 11

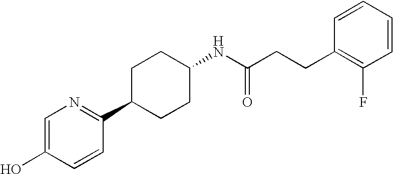

3-(2-fluorophenyl)-N-[trans-4-(5-hydroxypyridin-2-yl)cyclohexyl]propanamide 11-A: trans-N-Benzyl-4-[5-(benzyloxy)pyridin-2-yl]cyclohexanamine

A mixture of 4-[5-(Benzyloxy)pyridin-2-yl]cyclohexanone (3.2 g, 11 mmol) and benzylamine (3.7 ml, 34 mmol, 5-B) in methanol (70 ml) was stirred at room temperature overnight. After cooling to −30° C., to the mixture was added portionwise NaBH4 (400 mg, 11 mmol) and the resulting mixture was stirred at the same temperature for 1 hour. NH-gel (30 g) was added and the mixture was concentrated in vacuum. The residue was purified by a short clumn (NH-gel, 150 g), eluting with dichloromethane-methanol (20:1). After evaporation, the residue was crystallized from diisopropylether to afford the titled compound as a white solid. (2.8 g, 67%)

¹H NMR (CDCl₃) δ: 8.29 (d, J=2.9 Hz, 1H), 7.46-7.02 (m, 12H), 5.08 (s, 2H), 3.85 (s, 2H), 2.73-2.53 (m, 2H), 2.17-1.91 (m, 4H), 1.67-1.15 (m, 4H) ppm. (NH was not observed)

11-B: trans-6-(4-Aminocyclohexyl)pyridin-3-ol

A mixture of trans-N-Benzyl-4-[5-(benzyloxy)pyridin-2-yl]cyclohexanamine (2.8 g, 7.5 mmol) and 20% Pd(OH)₂—C (0.28 g) in methanol (30 ml) was stirred for 5 hours under hydrogen (4 kg/cm²). After filtration through a pad of celite, the filtrate was concentrated in vacuum. The solid was slurried with hexane and filtered to afford the titled compound. (1.40 g, 97%)

¹H NMR (d-DMSO) δ: 8.06-7.97 (m, 1H), 7.07-6.95 (m, 2H), 2.60-2.38 (m, 4H), 1.87-1.70 (m, 4H), 1.55-1.36 (m, 2H), 1.18-1.00 (m, 2H) ppm. (—OH and —NH₂ were not observed)

11-C: 3-(2-fluorophenyl)-N-[trans-4-(5-hydroxypyridin-2-yl)cyclohexyl]propanamide

The title compound was prepared from trans-6-(4-Aminocyclohexyl)pyridin-3-ol and 3-(2-fluorophenyl)propionic acid by the same manner as example 7-C.

¹H NMR (DMSO-d₆) δ: 9.61 (brs, 1H), 8.03-7.97 (m, 1H), 7.76 (d, J=7.9 Hz, 1H), 7.34-7.03 (m, 6H), 3.63-3.46 (m, 1H), 2.83 (t, J=7.6 Hz, 2H), 2.56-2.43 (m, 1H), 2.32 (t, J=7.6 Hz, 2H), 1.88-1.74 (m, 4H), 1.60-1.40 (m, 2H), 1.32-1.15 (m, 2H) ppm.

IR (KBr)vₘₐₓ: 3283, 2932, 1641, 1553, 1493, 1283, 1229, 750 cm⁻¹.

MS (ESI): 343.0 (M+H)⁺, 341.0 (M−H)⁻

### Example 12

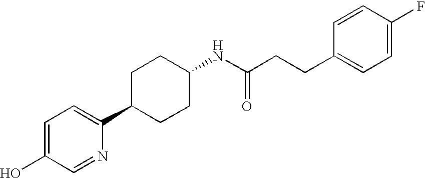

3-(4-fluorophenyl)-N-[trans-4-(5-hydroxypyridin-2-yl)cyclohexyl]propanamide

The title compound was prepared from 6-(trans-4-aminocyclohexyl)pyridin-3-ol and 3-(4-fluorophenyl)propionic acid by the same manner as example 7-C.

¹H NMR (DMSO-d₆) δ: 9.62 (brs, 1H), 8.05-7.98 (m, 1H), 7.71 (d, J=7.8 Hz, 1H), 7.28-7.18 (m, 2H), 7.14-7.02 (m, 4H), 3.64-3.40 (m, 1H), 2.79 (t, J=7.5 Hz, 2H), 2.62-2.40 (m, 1H), 2.33 (t, J=7.5 Hz, 2H) 1.91-1.73 (m, 4H), 1.60-1.39 (m, 2H), 1.34-1.10 (m, 2H) ppm.

MS (ESI): 343.17 (M+H)⁺, 341.14 (M−H)⁻

IR (KBr)vₘₐₓ: 3483, 3300, 2934, 1638, 1601, 1547, 1508, 1495, 1456, 1277, 1221, 1155, 1097, 976, 897, 833 cm⁻¹.

m.p. 195.4° C.

### Example 13

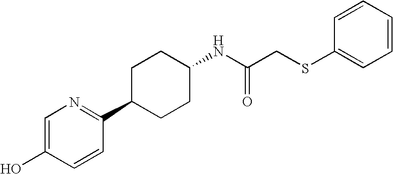

N-[trans-4-(5-hydroxypyridin-2-yl)cyclohexyl]-2-(phenylthio)acetamide

The title compound was prepared from 6-(trans-4-aminocyclohexyl)pyridin-3-ol and 2-(phenylthio)acetic acid by the same manner as example 7-C.

¹H NMR (DMSO-d₆) δ: 9.60 (brs, 1H), 8.07-7.98 (m, 2H), 7.40-7.28 (m, 4H), 7.23-7.15 (m, 1H), 7.06 (d, J=1.8 Hz, 1H), 3.61 (s, 2H), 3.59-3.48 (m, 1H), 2.62-2.40 (m, 1H), 1.90-1.76 (m, 4H), 1.58-1.40 (m, 2H), 1.35-1.17 (m, 2H) ppm.

IR (KBr)vₘₐₓ: 3329, 2930, 1645, 1537, 1279, 837, 741 cm⁻¹.

MS (ESI): 343.17 (M+H)⁺, 341.12 (M−H)⁻

The Synthetic Procedure of Example 14-Example 20

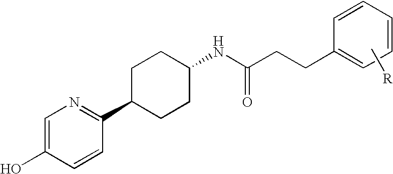

The compounds disclosed hereinafter were prepared according to the following procedure:

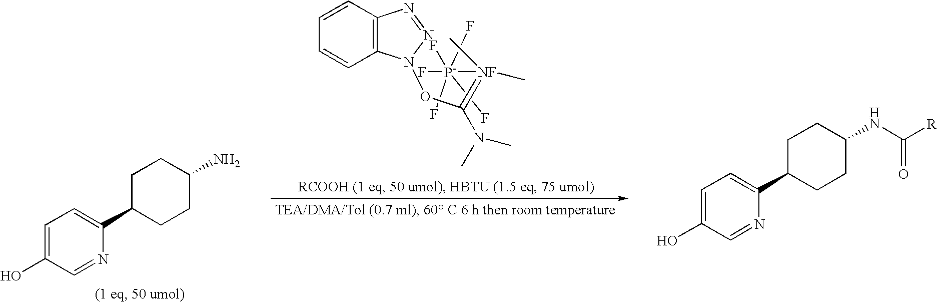

To acid (0.050 mmol) were added toluene (0.30 ml), 6-(trans-4-aminocyclohexyl)pyridin-3-ol (0.050 mmol) in 3.8%-triethylamine/dimethylacetamide (0.2 ml), O-benzotriazol-1-yl-N,N,N′,N′-tetramethyluronium hexafluorophosphate (0.075 mmol) in dimethylacetamide (0.20 ml). The resultant mixture was stirred at 60° C. for 6 hours and then stirred t at room temperature overnight. The mixture was evaporated. To the residue was added 1 M NH₃/MeOH (1 ml) and the resulting mixture was stirred at 40° C. for 2 hours, followed by evaporation of the volatiles. The residue was dissolved with MeOH (0.8 ml), which was loaded onto SCX-SPE cartridge (1 g/6 ml) preconditioned with MeOH (8 ml). The column was washed with MeOH (8 ml), and then eluted with 1 M NH₃/MeOH (5 ml). The mixture was concentrated to dryness and the crude material was purified with preparative LC/MS to afford the desired product.

### Example 14

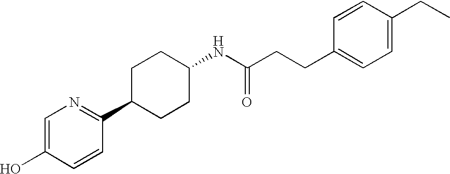

3-(4-ethylphenyl)-N-[trans-4-(5-hydroxypyridin-2-yl)cyclohexyl]propanamide

Observed MS (ESI) m/z 353.36 (M+H)⁺

### Example 15

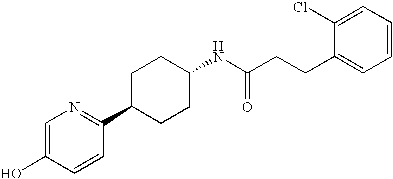

3-(2-chlorophenyl)-N-[trans-4-(5-hydroxypyridin-2-yl)cyclohexyl]propanamide

Observed MS (ESI) m/z 359.25 (M+H)⁺

### Example 16

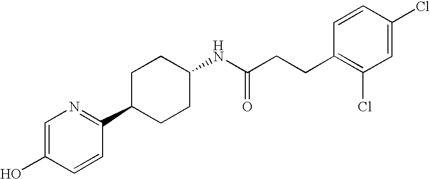

3-(2,4-dichlorophenyl)-N-[trans-4-(5-hydroxypyridin-2-yl)cyclohexyl]propanamide

Observed MS (ESI) m/z 393.21 (M+H)⁺

### Example 17

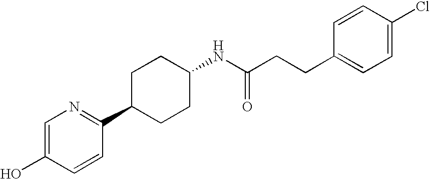

3-(4-chlorophenyl)-N-[trans-4-(5-hydroxypyridin-2-yl)cyclohexyl]propanamide

Observed MS (ESI) m/z 359.25 (M+H)⁺

### Example 18

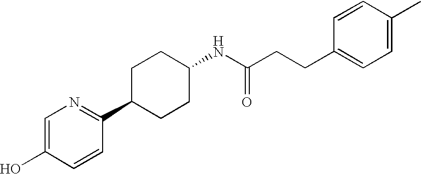

3-(4-methylphenyl)-N-[trans-4-(5-hydroxypyridin-2-yl)cyclohexyl]propanamide

Observed MS (ESI) m/z 339.34 (M+H)⁺

### Example 19

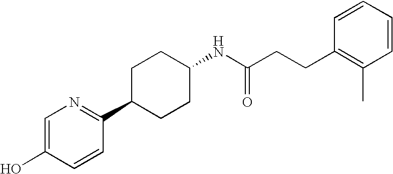

3-(2-methylphenyl)-N-[trans-4-(5-hydroxypyridin-2-yl)cyclohexyl]propanamide

Observed MS (ESI) m/z 339.34 (M+H)⁺

### Example 20

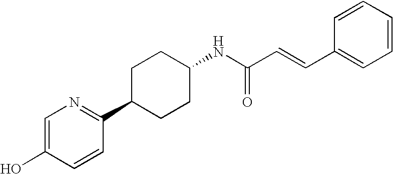

3-(2-methylphenyl)-N-trans-4-(5-hydroxypyridin-2-yl)cyclohexyl]propanamide

Observed MS (ESI) m/z 323.31 (M+H)⁺

### Example 21

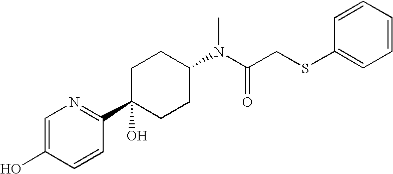

N-[cis-4-hydroxy-4-(5-hydroxypyridin-2-yl)cyclohexyl]-N-methyl-2-(phenylthio)acetamide

21-A: 6-[cis-1-hydroxy-4-(methylamino)cyclohexyl]pyridin-3-ol

The title compound was prepared from cis-1-[5-(benzyloxy)pyridin-2-yl]-4-(methylamino)cyclohexanol(4-A) by the same manner as example 7-B.

¹H NMR (DMSO-d₆) δ: 8.00 (d, J=2.8 Hz, 1H), 7.43 (d, J=8.6 Hz, 1H), 8.01 (dd, J=8.6, 2.8 Hz, 1H), 4.80 (brs, 1H), 2.62-2.16 (m, 5H), 1.97-1.33 (m, 8H) ppm. (—OH and —NH were not observed)

21-B: N-[cis-4-hydroxy-4-(5-hydroxypyridin-2-yl)cyclohexyl]-N-methyl-2-(phenylthio)acetamide

The title compound was prepared from 6-[cis-1-hydroxy-4-(methylamino)cyclohexyl]pyridin-3-ol and 2-(phenylthio)acetic acid by the same manner as example 7-C.

¹H NMR (DMSO-d₆) δ: 9.70 (brs, 1H), 8.01 (d, J=2.8 Hz, 1H), 7.50-7.27 (m, 5H), 7.23-7.10 (m, 2H), 4.98 (s, 1H), 4.38-4.25 (m, 0.5H), 4.06 (s, 1H), 3.99 (s, 1H), 3.88-3.74 (m, 0.5H), 2.93 (s, 1.5H), 2.75 (s, 1.5H), 2.15-1.85 (m, 4H), 1.66-1.28 (m, 4H) ppm.

IR (KBr)vₘₐₓ: 2920, 1587, 1481, 1265, 1089, 745 cm⁻¹.

MS (ESI): 373.0 (M+H)⁺, 370.9 (M−H)⁻

### Example 22

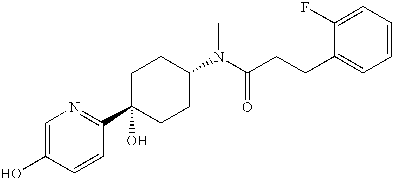

3-(2-fluorophenyl)-N-[cis-4-hydroxy-4-(5-hydroxypyridin-2-yl)cyclohexyl]-N-methylpropanamide

The title compound was prepared from 6-[cis-1-hydroxy-4-(methylamino)cyclohexyl]pyridin-3-ol and 3-(2-fluorophenyl)propionic acid by the same manner as example 7-C.

¹H NMR (DMSO-d₆) δ: 9.71 (brs, 11H), 8.07-8.00 (m, 11H), 7.50-7.09 (m, 6H), 4.98-4.95 (m, 1H), 4.47-4.30 and 3.80-3.68 (m, 1H)2.92-2.50 (m, 7H), 2.13-1.85 (m, 4H), 1.66-1.28 (m, 4H) ppm.

IR (KBr)vₘₐₓ: 3163, 1585, 1491, 1265, 1227, 1105, 766 cm⁻¹.

MS (ESI): 373.0 (M+H)⁺, 371.0 (M−H)⁻

The Synthetic Procedure of Example 23-Example 24

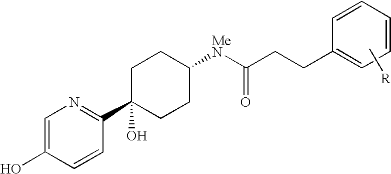

The compounds disclosed hereinafter were prepared according to the following procedure:

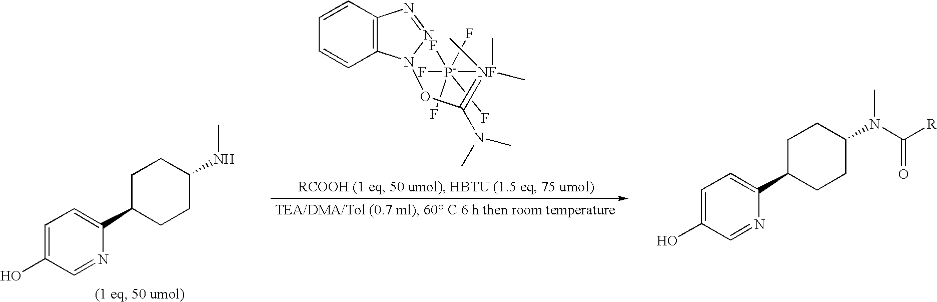

To acid (0.050 mmol) were added toluene (0.30 ml), 6-(trans-4-methylaminocyclohexyl)pyridin-3-ol (0.050 mmol) in 3.8%-triethylamine/dimethylacetamide (0.2 ml), O-benzotriazol-1-yl-N,N,N′,N′-tetramethyluronium hexafluorophosphate (0.075 mmol) in dimethylacetamide (0.20 ml). The resultant mixture was stirred at 60° C. for 6 hours and then stirred t at room temperature overnight. The mixture was evaporated. To the residue was added 1 M NH₃/MeOH (1 ml) and the resulting mixture was stirred at 40° C. for 2 hours, followed by evaporation of the volatiles. The residue was dissolved with MeOH (0.8 ml), which was loaded onto SCX-SPE cartridge (1 g/6 ml) preconditioned with MeOH (8 ml). The column was washed with MeOH (8 ml), and then eluted with 1 M NH₃/MeOH (5 ml). The mixture was concentrated to dryness and the crude material was purified with preparative LC/MS to afford the desired product.

### Example 23

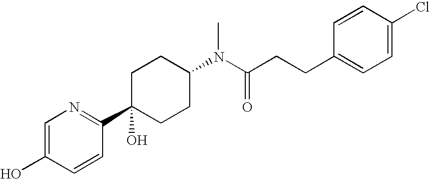

3-(4-chlorophenyl)-N-[cis-4-hydroxy-4-(5-hydroxypyridin-2-yl)cyclohexyl]-N-methylpropanamide

Observed MS (ESI) m/z 389.25 (M+H)⁺

### Example 24

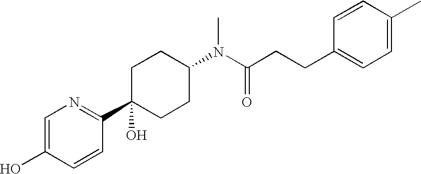

3-(4-methylphenyl)-N-[cis-4-hydroxy-4-(5-hydroxypyridin-2-yl)cyclohexyl]-N-methylpropanamide

Observed MS (ESI) m/z 369.35 (M+H)⁺

### Example 25

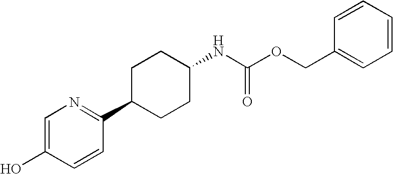

trans-Benzyl 4-(5-hydroxypyridin-2-yl)cyclohexylcarbamate

25-A: trans-Benzyl 6-(4-{[(benzyloxy)carbonyl]amino}cyclohexyl)pyridin-3-yl Carbonate

Benzyl chloroformate (0.040 ml, 0.28 mmol) was added dropwise to a solution of trans-6-(4-aminocyclohexyl)pyridin-3-ol (41 mg, 0.2 mmol, 11-B) and sodium carbonate (29 mg, 0.28 mmol) in MeOH—H₂O (0.5 ml-2.0 ml) at 0° C. The mixture was stirred at room temperature for 5 hours. The mixture was evaporated in vacuum and the residue was extracted with dichloromethane (5.0 ml×2). The combined extracts were dried over MgSO4 and concentarated in vacuum to afford the titled compound as a yellow solid. (45 mg, 47%)

MS (ESI): 461.29 (M+H)⁺

25-B: trans-Benzyl 4-(5-hydroxypyridin-2-yl)cyclohexylcarbamate

To a solution of trans-benzyl 6-(4-{[(benzyloxy)carbonyl]amino}cyclohexyl)pyridin-3-yl carbonate (45 mg, 0.098 mmol) in methanol (3.0 ml) was added 1 M-NaOH aq. (0.5 ml) and the mixture was stirred at room temperature for 1.5 hours. The mixture was evaporated in vacuum, and the residue was treated with H₂O (1.0 ml). The whole was extracted with dichloromethane (5.0 ml×2). The combined extracts were dried over MgSO₄ and concentarated in vacuum. The residue was purified by PTLC (dichloromethane/methanol=12/1 as eluent) to afford the titled compound as a white solid. (13 mg, 42%)

¹H NMR (DMSO-d₆) δ: 8.06-7.97 (m, 1H), 7.45-7.19 (m, 5H), 7.15-7.08 (m, 2H), 5.01 (s, 2H), 2.60-2.40 (m, 2H), 2.01-1.73 (m, 4H), 1.63-1.40 (m, 2H), 1.40-1.16 (m, 2H) ppm. (—OH and —NH were not observed)

IR (KBr)vₘₐₓ: 3367, 2939, 2517, 1699, 1570, 1306, 1283, 1265, 1132, 1047, 696 cm⁻¹.

MS (ESI): 327.17 (M+H)⁺, 325.11 (M−H)⁻

### Example 26

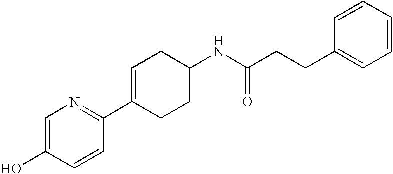

N-[4-(5-Hydroxypyridin-2-yl)cyclohex-3-en-1-yl]-3-phenylpropanamide

To a stirred solution of (diethylamino)sulfur trifluoride (0.19 ml, 1.4 mmol) in dichloromethane (4.0 ml) was added a suspension of N-[4-hydroxy-4-(5-hydroxypyridin-2-yl)cyclohexyl]-3-phenylpropanamide (150 mg, 0.44 mmol, 1-C) in dichloromethane (4.0 ml) at −78° C. The mixture was stirred at −78° C. for 1 hour and at 0° C. for 4 hours. The mixture was quenched with sat. K₂CO₃ aq. The whole was extracted with dichloromethane (5.0 ml×2). The combined extracts were dried over MgSO₄ and concentarated in vacuum. The residue was purified by PTLC (dichloromethane/methanol=20/1 as eluent) to afford the titled compound as a white solid (5 mg, 4%).

¹H NMR (CD₃OD) δ: 8.00 (d, J=3.0 Hz, 1H), 7.96-7.84 (m, 1H), 7.40-7.07 (m, 7H), 6.32-6.20 (m, 1H), 4.04-3.83 (m, 1H), 2.99-2.83 (m, 2H), 2.64-2.35 (m, 5H), 2.12-1.81 (m, 2H), 1.72-1.50 (m, 1H) ppm. (—OH was not observed)

IR (KBr)vₘₐₓ: 3227, 2934, 1647, 1595, 1544, 1481, 1445, 1273, 1128, 743, 704 cm⁻¹.

MS (ESI): 323.13 (M+H)⁺, 321.03 (M−H)⁻

### Example 27

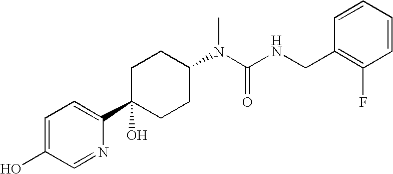

N′-(2-fluorobenzyl)-N-[cis-4-hydroxy-4-(5-hydroxypyridin-2-yl)cyclohexyl]-N-methylurea

27-A: N-{cis-4-[5-(benzyloxy)pyridin-2-yl]-4-hydroxycyclohexyl}-N′-(2-fluorobenzyl)-N-methylurea

To a solution of cis-1-[5-(benzyloxy)pyridin-2-yl]-4-(methylamino)cyclohexanol (0.99 g, 3.2 mmol, 6-A) and triethylamine (0.44 ml, 3.2 mmol) in dichloromethane (12 ml) was added 2-fluorobenzyl isocyanate (0.48 g, 3.2 mmol) and the mixture was stirred at room temperature for 5 hours. The mixture was evaporated in vacuum. The residue was purified by column chromatography on silica gel (dichloromethane: methanol=40:1 as eluent) to afford the titled compound as a white solid. (1.4 g, 97%)

¹H NMR (DMSO-d₆) δ: 8.25 (d, J=2.3 Hz, 1H), 7.55-7.16 (m, 9H), 7.16-6.95 (m, 2H), 5.11 (s, 2H), 4.85-4.70 (m, 1H), 4.51 (d, J=5.8 Hz, 2H), 4.40-4.25 (m, 1H), 2.82 (s, 3H), 2.15-1.50 (m, 8H) ppm. (—OH was not observed)

MS (ESI): 464.10 (M+H)⁺

27-B: N′-(2-fluorobenzyl)-N-[cis-4-hydroxy-4-(5-hydroxypyridin-2-yl)cyclohexyl]-N-methylurea

A mixture of N-{cis-4-[5-(benzyloxy)pyridin-2-yl]-4-hydroxycyclohexyl}-N′-(2-fluorobenzyl)-N-methylurea (1.4 g, 3.1 mmol) in methanol (15 ml) was hydrogenated using 10% Pd/C (0.36 g) on H₂ (1 atm) at room temperature for 1 day. The mixture was filtered off through a pad of celite and the filtrate was concentrated in vacuum. The residue was purified by column chromatography on silica gel (dichloromethane: methanol=20:1 as eluent) to give the white solid. This solid was recrystallized from ethanol to afford the titled compound as a white solid. (0.51 g, 45%)

¹H NMR (DMSO-d₆)δ: 9.69 (brs, 1H), 8.09-7.92 (m, 1H), 7.51-7.41 (m, 1H), 7.38-6.99 (m, 5H), 6.94-6.75 (m, 1H), 4.93 (s, 1H), 4.28 (d, J=5.4 Hz, 2H), 4.09-3.89 (m, 1H), 2.73 (s, 3H), 2.10-1.78 (m, 4H), 1.67-1.47 (m, 2H), 1.46-1.25 (m, 2H) ppm.

MS (ESI): 374.22 (M+H)⁺, 372.18 (M−H)⁻

IR (KBr)vₘₐₓ: 3393, 3117, 2934, 1599, 1572, 1529, 1489, 1458, 1327, 1271, 1225, 1173, 1130, 1043, 756, 609 cm⁻¹.

### Example 28

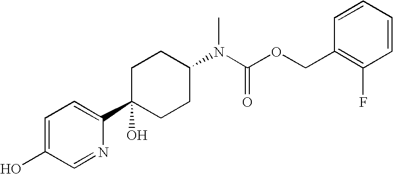

2-fluorobenzyl [cis-4-hydroxy-4-(5-hydroxypyridin-2-yl)cyclohexyl]methylcarbamate

A mixture of 6-[cis-1-hydroxy-4-(methylamino)cyclohexyl]pyridin-3-ol (0.20 g, 0.90 mmol, 21-A) and 1-({[(2-fluorobenzyl)oxy]carbonyl}oxy)pyrrolidine-2,5-dione (0.72 g, 2.7 mmol) in THF-sat. NaHCO₃ aq. (5.0 ml-12 ml) was stirred at room temperature overnight. The mixture was diluted with H₂O and extracted with ethyl acetate (20 ml×2). The combined extracts were dried over MgSO₄ and concentrated in vacuum to afford the colorless oil. A mixture of this oil, methanol (30 ml), and 1 M NaOH (5.0 ml) was stirred for 3 hours at room temperature. After evaporated in vacuum, the residue was neutralized with 2 M HCl aq. The whole was extracted with dichloromethane (20 ml×2). The combined extracts were washed with sat. NaHCO₃ aq., dried over MgSO₄ and concentrated in vacuum. The residue was purified by column chromatography on silica gel (dichloromethane:methanol=20:1 as eluent) to afford the titled compound as a white solid (0.23 g, 67%).

¹H NMR (DMSO-d₆) δ: 9.78 (brs, 1H), 8.15-8.05 (m, 1H), 7.63-7.40 (m, 3H), 7.40-7.12 (m, 3H), 5.20 (s, 2H), 5.04 (s, 1H), 4.14-3.84 (m, 1H), 2.85 (s, 3H), 2.17-1.88 (m, 4H), 1.77-1.38 (m, 4H) ppm.

MS (ESI): 375.0 (M+H)⁺, 373.0 (M−H)⁻

IR (KBr)vₘₐₓ: 3200, 2947, 1651, 1497, 1433, 1416, 1329, 1269, 1234, 1190, 1169, 1117, 1034, 1005, 945, 762 cm⁻¹.

### Example 29

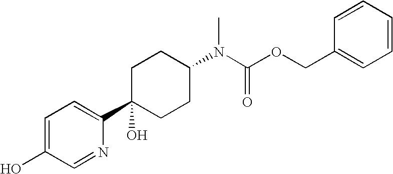

Benzyl [cis-4-hydroxy-4-(5-hydroxypridin-2-yl)cyclohexyl]methylcarbamate

To a mixture of 6-[cis-1-hydroxy-4-(methylamino)cyclohexyl]pyridin-3-ol (0.040 g, 0.18 mmol) and sodium carbonate (0.063 g, 0.59 mmol) in methanol-H₂O (1.0 ml-4.0 ml) was added benzyl chloroformate (0.085 ml, 0.59 mmol) and the mixture was stirred at room temperature for 6 hours. The mixture was diluted with H₂O and evaporated in vacuum. The residue was extracted with dichloromethane (10 ml×2). The combined extracts were dried over MgSO₄ and concentrated in vacuum to afford the colorless oil. A mixture of this oil, methanol (5 ml), and 1 M NaOH (0.8 ml) was stirred at room temperature for 2 hours. After the mixture was evaporated in vacuum, the residue was neutralized with 2M HCl aq. The whole was extracted with dichloromethane (5.0 ml×2). The combined extracts were washed with sat. NaHCO₃ aq., dried over MgSO₄ and concentrated in vacuum. The residue was purified by PTLC (dichloromethane:methanol=12:1 as eluent) to afford the titled compound as a white solid (0.032 g, 56%).

¹H NMR (CDCl₃) δ: 8.24-8.13 (m, 1H), 7.51-7.10 (m, 7H), 5.17 (s, 2H), 4.32-3.98 (m, 1H), 2.88 (s, 3H), 2.21-1.94 (m, 2H), 1.94-1.47 (m, 6H) ppm. (—OH was not observed)

MS (ESI): 357.14 (M+H)⁺, 355.08 (M−H)⁻

IR (KBr)vₘₐₓ: 3460, 3219, 1670, 1491, 1445, 1410, 1356, 1321, 1263, 1217, 1157, 1113, 1040, 1007, 766 cm⁻¹.

### Example 30

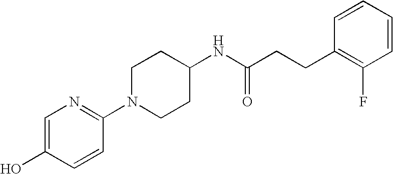

3-(2-fluorophenyl)-N-[1-(5-hydroxypyridin-2-yl)piperidin-4-yl]propanamide 30-A: 5-(Benzyloxy)-2-bromopyridine

To a suspension of NaH (60% in oil, 0.28 g, 6.9 mmol) in THF (6.0 ml) was added 6-bromopyridin-3-ol (1.0 g, 5.8 mmol) at 0° C. The mixture was stirred at 0° C. for 30 min and at room temperature for additional 30 min. To this mixture was added a solution of benzylbromide (1.1 g, 6.3 mmol) in DMSO (6.0 ml) slowly at room temperature and the mixture was stirred at room temperature overnight. Sat. NaH₂PO₄ aq. was slowly added to the mixture and the organic layer was separated. The aqueous layer was extracted with ethyl acetate (10 ml×3). The combined extracts were washed with brine, dried over MgSO₄, and evaporated in vacuum. The residue was purified by column chromatography on silica gel (hexane:ethyl acetate=50:1 as eluent) to afford the titled compound as colorless oil. (1.2 g, 79%)

¹H NMR (DMSO-d₆) δ: 8.20 (d, J=2.9 Hz, 1H), 7.56 (d, J=8.8 Hz, 1H), 7.50-7.32 (m, 6H), 5.19 (s, 2H) ppm.

30-B: tert-butyl {1-[5-(benzyloxy)pyridin-2-yl]piperidin-4-yl}carbamate

A mixture of 5-(Benzyloxy)-2-bromopyridine (2.1 g, 8.0 mmol) and tert-butyl piperidin-4-ylcarbamate (6.4 g, 32 mmol) in DMSO (120 ml) was stirred at 150° C. for 24 hours and poured onto water (100 ml). The whole was extracted with ethyl acetate (75 ml×2). The combined extracts were washed with brine, dried over MgSO₄, and evaporated in vacuum. The residue was purified by column chromatography on silica gel (hexane:ethyl acetate=3:1 as eluent) to afford the titled compound. (1.0 g, 33%)

¹H NMR (CDCl₃) δ: 7.97 (d, J=3.1 Hz, 1H), 7.45-7.28 (m, 5H), 6.62 (dd, J=9.2 Hz, J=3.1 Hz, 1H), 6.64 (d, J=9.2 Hz, 1H), 5.01 (s, 2H), 4.53-4.38 (m, 1H), 4.08-3.97 (m, 2H), 3.75-3.57 (m, 1H), 2.95-2.84 (m, 2H), 2.09-1.98 (m, 2H), 1.66-1.40 (m, 11H) ppm.

30-C: N-{1-[5-(benzyloxy)pyridin-2-yl]piperidin-4-yl}-3-(2-fluorophenyl)propanamide

A mixture of tert-butyl {1-[5-(benzyloxy)pyridin-2-yl]piperidin-4-yl}carbamate (0.31 g, 0.81 mmol) and 4 M-HCl (ethyl acetate soln, 4 ml, 16 mmol) in ethyl acetate (8 ml) was stirred at room temperature for 4 hours and the solvent was removed in vacuum. The residue was diluted with DMF (15 ml). To the mixture were added 3-(2-fluorophenyl)propanoic acid (0.14 g, 0.81 mmol), WSC (0.19 g, 1.00 mmol), HOBt (0.15 g, 1.00 mmol), and triethylamine (0.23 ml, 1.6 mmol). The reaction mixture was stirred at room temperature for 24 hours and quenched with water (20 ml). The whole was extracted with ethyl acetate (20 ml×2). The combined extracts were washed with brine, dried over MgSO₄, and evaporated in vacuum. The residue was purified by column chromatography on silica gel (dichloromethane: methanol=30:1 as eluent) to afford the titled compound. (0.11 g, 32%)

¹H NMR (CDCl₃) δ: 7.96 (d, J=2.9 Hz, 1H), 7.45-6.95 (m, 10H), 6.62 (d, J=9.2 Hz, 1H), 5.32 (d, J=7.9 Hz, 1H), 5.02 (s, 2H), 4.07-3.88 (m, 3H), 2.99 (t, J=7.5 Hz, 1H), 2.95-2.82 (m, 2H), 2.46 (t, J=7.5 Hz, 2H), 1.98-1.86 (m, 2H), 1.44-1.26 (m, 2H) ppm.

30-D: 3-(2-fluorophenyl)-N-[1-(5-hydroxypyridin-2-yl)piperidin-4-yl]propanamide

A mixture of N-{1-[5-(benzyloxy)pyridin-2-yl]piperidin-4-yl}-3-(2-fluorophenyl)propanamide (0.11 g, 0.26 mmol) and 10%-Pd-C (20 mg) was stirred for 7 hours under hydrogen (4 kg/cm²). After filtration by a pad of celite, the filtrate was concentrated in vacuum. The residue was purified by preparative TLC to afford the titled compound. (11 mg, 12%)

¹H NMR (DMSO-d₆) δ: 8.94 (brs, 1H), 7.80 (d, J=7.5 Hz, 1H), 7.71 (d, J=2.4 Hz, 1H), 7.30-7.08 (m, 4H), 7.03 (dd, J=9.0 Hz, J=3.1 Hz, 1H), 6.72 (d, J=9.0 Hz, 1H), 4.00-3.88 (m, 2H), 3.82-3.65 (m, 1H), 2.87-2.70 (m, 4H), 2.39-2.30 (m, 2H), 1.76-1.64 (m, 2H), 1.39-1.24 (m, 2H) ppm.

MS (ESI): 344.0 (M+H)⁺, 341.9 (M−H)⁻

### Example 31

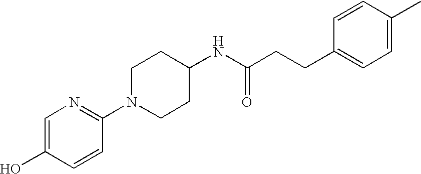

N-[1-(5-hydroxypyridin-2-yl)piperidin-4-yl]-3-(4-methylphenyl)propanamide 31-A: N-{1-[5-(benzyloxy)pyridin-2-yl]piperidin-4-yl}-3-(4-methylphenyl)propanamide

The title compound was prepared from tert-butyl {1-[5-(benzyloxy)pyridin-2-yl]piperidin-4-yl}carbamate by the same manner as example 30-C.

¹H NMR (CDCl₃) δ: 7.97 (d, J=2.9 Hz, 1H), 7.45-7.29 (m, 5H), 7.18 (dd, J=9.2 Hz, J=2.9 Hz, 1H), 7.14-7.06 (m, 4H), 6.62 (d, J=9.2 Hz, 1H), 5.18 (d, J=7.5 Hz, 1H), 5.02 (s, 2H), 4.03-3.88 (m, 3H), 2.99-2.85 (m, 4H), 2.43 (t, J=7.5 Hz, 2H), 2.30 (s, 3H), 1.98-1.88 (m, 2H), 1.43-1.28- (m, 2H) ppm.

31-B: N-[1-(5-hydroxypyridin-2-yl)piperidin-4-yl]-3-(4-methylphenyl)propanamide

The title compound was prepared from N-{1-[5-(benzyloxy)pyridin-2-yl]piperidin-4-yl}-3-(4-methylphenyl)propanamide by the same manner as example 30-D.

¹H NMR (DMSO-d₆) δ: 8.94 (brs, 1H), 7.75 (d, J=7.5 Hz, 1H), 7.71 (d, J=2.9 Hz, 1H), 7.10-7.00 (m, 5H), 6.72 (d, J=9.0 Hz, 1H), 3.99-3.88 (m, 2H), 3.78-3.64 (m, 1H), 2.83-2.70 (m, 4H), 2.35-2.26 (m, 2H), 2.25 (s, 3H), 1.76-1.64 (m, 2H), 1.39-1.24 (m, 2H) ppm.

IR (KBr)vₘₐₓ: cm⁻¹.

MS (ESI): 340.0 (M+H)⁺, 338.0 (M−H)⁻

1. A compound of the formula (I): 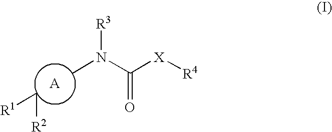
wherein R¹ represents 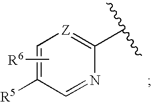
R⁵ represents a hydroxy group or an alkylsulfonylamino group having from 1 to 6 carbon atoms;
R⁶ and R⁷ independently represents a hydrogen atom, a halogen atom, an alkyl group having from 1 to 6 carbon atoms, an alkenyl group having from 2 to 6 carbon atoms, an alkoxy group having from 1 to 6 carbon atoms or, when Z represents a carbon atom and R⁶ is ortho to Z, R⁶ and Z taken together may form a fused phenyl group or a saturated or partially unsaturated cyclic ring having from 4 to 7 carbon atoms;
V represents an alkylene group having from 1 to 2 carbon atoms, imino, imino substituted with an alkyl group having from 1 to 6 carbon atoms, an oxygen atom or a sulfur atom;
W represents a carbon atom or a nitrogen atom;
Z represents a carbon atom or a nitrogen atom;
with the proviso that W and Z do not simultaneously represent a carbon atom;
R² represents a hydrogen atom or a hydroxy group or R² forms a covalent bond with ring a:
R³ represents a hydrogen atom or an alkyl group having from 1 to 6 carbon atoms:
A represents a cycloalkylene group having from 3 to 10 carbon atoms or a heterocyclic group having from 4 to 10 atoms;
X represents a covalent bond, an alkylene group having from 1 to 3 carbon atoms, an alkenylene group having from 2 to 3 carbon atoms, a heteroalkylene group having from 2 to 3 atoms, wherein one of said atoms is replaced by a sulfur atom, an oxygen atom, imino, imino substituted with an alkyl group having from 1 to 6 carbon atoms or a sulfonyl group, a cycloalkylene group having from 3 to 10 carbon atoms or a heterocyclic group having from 4 to 10 atoms;
R⁴ represents an aryl group having from 6 to 10 carbon atoms, a heteroaryl group having from 5 to 10 atoms;
said alkylene groups, alkenylene groups, heteroalkylene groups, cycloalkylene groups and heterocyclic groups are unsubstituted or are substituted by at least one substituent selected from the group consisting of substituents α;
said aryl groups having from 6 to 10 carbon atoms and said heteroaryl groups having from 5 to 10 atoms are unsubstituted or are substituted by at least one substituent selected from the group consisting of substituents β;
said substituents α are selected from the group consisting of alkyl groups having from 1 to 6 carbon atoms, cyano groups, alkanoylamino groups having from 1 to 7 carbon atoms, oxo groups or aryl groups having from 6 to 10 carbon atoms defined above;
said substituents β are selected from the atom consisting of halogen atoms, alkyl groups having from 1 to 6 carbon atoms, alkoxy groups having from 1 to 6 carbon atoms, haloalkyl groups having from 1 to 6 carbon atoms, alkylthio groups having from 1 to 6 carbon atoms, alkanoyl groups having from 1 to 7 carbon atoms, hydroxy groups, cyano groups, aryl groups having from 6 to 10 carbon atoms defined above or heteroaryl groups having from 5 to 10 atoms defined above;
with the proviso that said aryl groups having from 6 to 10 carbon atoms and said heteroaryl groups having from 5 to 10 atoms in said substituents α and β are not substituted by an aryl group having from 6 to 10 carbon atoms or heteroaryl groups having from 5 to 10 atoms; and
or a pharmaceutically acceptable ester of such compound;
or a pharmaceutically acceptable salt thereof.

2. A compound according to Claim 1, wherein:
R¹ represents
Z represents a carbon atom;
R⁵ represents a hydroxy group; and
R⁶ represents a hydrogen atom, a halogen atom or an alkyl group having from 1 to 6 carbon atoms.

3. A compound according to Claim 1, wherein R² represents a hydrogen atom or a hydroxy group.

4. A compound according to Claim 1, wherein R³ represents a hydrogen atom or a methyl group.

5. A compound according to Claim 1, wherein A represents a substituted or unsubstituted cycloalkylene group having from 3 to 8 carbon atoms, or an heterocyclic group having from 4 to 8 atoms which consists of at least one carbon atom and from 1 to 2 nitrogen atoms wherein the substituent is at least one group selected from alkyl groups having from 1 to 6 carbon atoms or oxo groups.

6. A compound according to Claim 1, wherein A represents a cyclohexyl group, a cyclohexenyl group or a piperidinyl group.

7. A compound according to Claim 1, wherein A represents a cyclohexyl group.

8. A compound according to Claim 1, wherein X represents an alkylene group having from 1 to 3 carbon atoms, a heteroalkylene group having from 2 to 3 atoms, wherein one of said atoms is replaced by a sulfur atom or an oxygen atom

9. A compound according to Claim 1, wherein X represents an alkylene group having from 1 to 3 carbon atoms or a heteroalkylene group having from 2 to 3 atoms, wherein one of said atoms is replaced by a sulfur atom.

10. A compound of formula (Ia) 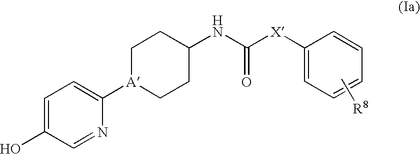
wherein
A′ represents CH, C(OH), or N;
X′ represents ethylene, oxymethylene, methyleneoxy, or methylenethio; and
R⁸ represents one or two groups independently selected from hydrogen atoms, alkyl groups having from 1 to 6 carbon atoms and halogen atoms or a pharmaceutically acceptable ester of such compound;
or a pharmaceutically acceptable salt thereof.

11. A compound according to Claim 1, wherein R⁴ represents a phenyl group, optionally substituted by at least one substituent selected from the group consisting of halogen atoms or alkyl groups having from 1 to 6 carbon atoms.

12. A compound according to Claim 1 selected from:
N-[cis-4-Hydroxy-4-(5-hydroxypyridin-2-yl)cyclohexyl]-3-phenylpropanamide hydrochloride;
3-(4-Chlorophenyl)-N-[cis-4-hydroxy-4-(5-hydroxypyridin-2-yl)cyclohexyl] propanamide;
N-[cis-4-Hydroxy-4-(5-hydroxypyridin-2-yl)cyclohexyl]-N-methyl-3-phenylpropanamide;
N-[trans-4-(5-Hydroxypyridin-2-yl)cyclohexyl]-3-phenylpropanamide hydrochloride;
N-[trans-4-(5-Hydroxypyridin-2-yl)cyclohexyl]-N-methyl-3-phenylpropanamide hydrochloride;
3-(2,4-dichlorophenyl)-N-[cis-4-hydroxy-4-(5-hydroxypyridin-2-yl)cyclohexyl]propanamide;
N-[cis-4-hydroxy-4-(5-hydroxypyridin-2-yl)cyclohexyl]-3-(4-methylphenyl)propanamide;
3-(2-fluorophenyl)-N-[cis-4-hydroxy-4-(5-hydroxypyridin-2-yl)cyclohexyl]propanamide;
3-(2-fluorophenyl)-N-[trans-4-(5-hydroxypyridin-2-yl)cyclohexyl]propanamide;
3-(4-fluorophenyl)-N-[trans-4-(5-hydroxypyridin-2-yl)cyclohexyl]propanamide;
N-[trans-4-(5-hydroxypyridin-2-yl)cyclohexyl]-2-(phenylthio)acetamide;
3-(4-ethylphenyl)-N-[trans-4-(5-hydroxypyridin-2-yl)cyclohexyl]propanamide;
3-(2-chlorophenyl)-N-[trans-4-(5-hydroxypyridin-2-yl)cyclohexyl]propanamide;
3-(4-chlorophenyl)-N-[trans-4-(5-hydroxypyridin-2-yl)cyclohexyl]propanamide;
3-(4-methylphenyl)-N-[trans-4-(5-hydroxypyridin-2-yl)cyclohexyl]propanamide;
3-(2-fluorophenyl)-N-[cis-4-hydroxy-4-(5-hydroxypyridin-2-yl)cyclohexyl]-N-methylpropanamide;
N-[4-(5-Hydroxypyridin-2-yl)cyclohex-3-en-1-yl]-3-phenylpropanamide;
2-fluorobenzyl [cis-4-hydroxy-4-(5-hydroxypyridin-2-yl)cyclohexyl]methylcarbamate;
benzyl [cis-4-hydroxy-4-(5-hydroxypyridin-2-yl)cyclohexyl]methylcarbamate;
3-(2-fluorophenyl)-N-[1-(5-hydroxypyridin-2-yl)piperidin-4-yl]propanamide; and
N-[1-(5-hydroxypyridin-2-yl)piperidin-4-yl]-3-(4-methylphenyl)propanamide;
or a pharmaceutically acceptable salt thereof.

13. A pharmaceutical composition for the treatment of disease conditions caused by overactivation of NMDA NR2B receptor, in a mammalian subject, which comprises a therapeutically effective amount of a compound according to claim 1, or a pharmaceutically acceptable ester of such compound, or a pharmaceutically acceptable salt thereof, and a suitable pharmaceutically acceptable carrier.

14. A method for the treatment of disease conditions caused by overactivation of NMDA NR2B receptor, in a mammalian subject, which comprises administering to said subject a therapeutically effective amount of a compound according to claim 1, or a pharmaceutically acceptable ester of such compound, or a pharmaceutically acceptable salt thereof.

15. A pharmaceutical composition for the treatment of disease conditions caused by overactivation of NMDA NR2B receptor, in a mammalian subject, which comprises a therapeutically effective amount of a compound according to claim 10, or a pharmaceutically acceptable ester of such compound, or a pharmaceutically acceptable salt thereof, and a suitable pharmaceutically acceptable carrier.

16. A pharmaceutical composition for the treatment of disease conditions caused by overactivation of NMDA NR2B receptor, in a mammalian subject, which comprises a therapeutically effective amount of a compound according to claim 10, or a pharmaceutically acceptable ester of such compound, or a pharmaceutically acceptable salt thereof, and a suitable pharmaceutically acceptable carrier.
